(19) United States
(12) Patent Application Publication
(10) Pub. No.: US 2025/0204862 A1
(43) Pub. Date: Jun. 26, 2025

(54) DYNAMICALLY HANDLING SUBSTANCE INTERFERENCE WITH ANALYTE SENSOR SYSTEMS

(71) Applicant: Dexcom, Inc., San Diego, CA (US)

(72) Inventors: Hifza NAJIB, San Diego, CA (US); Daniel CHERNAVVSKY, San Diego, CA (US); Abdulrahman JBAILY, San Diego, CA (US); Abdullah Zafar ABBASI, San Diego, CA (US)

(21) Appl. No.: 18/981,282

(22) Filed: Dec. 13, 2024

Related U.S. Application Data
(60) Provisional application No. 63/614,449, filed on Dec. 22, 2023.

(51) Int. Cl. A61B 5/00; A61B 5/145; G16H 10/60

(52) U.S. Cl. CPC A61B 5/7203; A61B 5/14532; A61B 5/7267; G16H 10/60

(57) ABSTRACT

In some aspects, a method of dynamically handling substance interference includes detecting an administration of a substance to a user of an analyte sensor system. The method also includes identifying the substance as an interferent with the analyte sensor system based on information related to the administration of the substance. The method also includes, responsive to the identifying, generating an interference effect of the substance on the analyte sensor system based on the information related to the administration of the substance. The method also includes determining an interference response based on the interference effect. The method also includes executing the interference response in relation to the analyte sensor system.

### CROSS-REFERENCE TO RELATED APPLICATIONS

This application claims priority to and benefit of U.S. Provisional Patent Application No. 63/614,449, filed Dec. 22, 2023, which is hereby expressly incorporated by reference herein in its entirety as if fully set forth below and for all applicable purposes.

### TECHNICAL FIELD

The present disclosure relates generally to medical devices such as analyte sensors, and more particularly, but not by way of limitation, to systems, devices, and methods for dynamically handling substance interference with analyte sensor systems.

### INTRODUCTION

Diabetes is a metabolic condition relating to the production or use of insulin by the body. Insulin is a hormone that allows the body to use glucose for energy, or store glucose as fat.

When a person eats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to several health problems, such as cardiovascular disease, cataract and other eye problems, nerve damage (neuropathy), and kidney damage. Hyperglycemia can also lead to acute problems, such as diabetic ketoacidosis—a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

Diabetes conditions are sometimes referred to as “Type 1” and “Type 2.” A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

Blood sugar concentration levels may be monitored with an analyte sensor, such as a continuous glucose monitor. A continuous glucose monitor may provide the wearer (patient) with information such as an estimated blood glucose level, a trend of estimated blood glucose levels, etc. However, the accuracy of such information is sometimes negatively impacted by interference from medication or other sources. This interference can be especially problematic in dynamic, fast-paced environments such as hospitals, urgent care facilities, emergency rooms, and hospice facilities that require rapid decision making and analysis.

This Background is provided to introduce a brief context for the Summary and Detailed Description that follow. This Background is not intended to be an aid in determining the scope of the claimed subject matter nor be viewed as limiting the claimed subject matter to implementations that solve any or all of the disadvantages or problems presented above.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One general aspect includes a method of dynamically handling substance interference. The method includes detecting an administration of a substance to a user of an analyte sensor system. The method also includes identifying the substance as an interferent with the analyte sensor system based on information related to the administration of the substance. The method also includes, responsive to the identifying, generating an interference effect of the substance on the analyte sensor system based on the information related to the administration of the substance. The method also includes determining an interference response based on the interference effect. The method also includes executing the interference response in relation to the analyte sensor system.

Another general aspect includes a system for dynamically handling substance interference. The system includes a memory having executable instructions and a processor in communication with the memory. The processor is configured to execute the instructions to detect an administration of a substance to a user of an analyte sensor system, identify the substance as an interferent with the analyte sensor system based on information related to the administration of the substance and, responsive to the identification, generate an interference effect of the substance on the analyte sensor system based on the information related to the administration of the substance. The processor is further configured to execute the instructions to determine an interference response based on the interference effect and execute the interference response in relation to the analyte sensor system.

Another general aspect includes a computer-program product including a non-transitory computer-usable medium. The non-transitory computer-usable medium has computer-readable program code embodied therein. The computer-readable program code is adapted to be executed to implement a method. The method includes detecting an administration of a substance to a user of an analyte sensor system. The method also includes identifying the substance as an interferent with the analyte sensor system based on information related to the administration of the substance. The method also includes, responsive to the identifying, generating an interference effect of the substance on the analyte sensor system based on the information related to the administration of the substance. The method also includes determining an interference response based on the interference effect. The method also includes executing the interference response in relation to the analyte sensor system.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document.

FIG. 1A illustrates an example of a health monitoring system, in accordance with certain aspects.

FIG. 1B illustrates an example analyte sensor system including an example continuous analyte sensor(s) with sensor electronics, in accordance with certain aspects.

FIG. 2 illustrates example inputs and example metrics that are generated based on the inputs in accordance with certain aspects.

FIG. 3 illustrates an example of a computing environment for implementing an interference detection and response (IDR) management system, in accordance with certain aspects.

FIG. 4 illustrates an example of a process for detecting and dynamically handling substance interference, in accordance with certain aspects.

FIG. 5 is a graph illustrating data that can be generated, for example, as part generating an interference effect, in accordance with certain aspects.

FIG. 6 illustrates an example of a process for implementing algorithmic compensation of analyte sensor measurements, in accordance with certain aspects.

FIG. 7 is a block diagram depicting a computer system configured for detecting and dynamically handling substance interference.

FIG. 8 is a flow diagram depicting a process for training machine learning models.

### DETAILED DESCRIPTION

Management of diabetes can present complex challenges for patients, clinicians, and caregivers, as a confluence of many factors can impact a patient's glucose level and glucose trends. To assist patients with better managing this condition, portable or wearable medical devices (e.g., sensors and other types of monitoring and diagnostic devices) as well as a variety of diabetes intervention software applications (hereinafter “applications”) have been developed by various providers.

Interstitial fluid (ISF) is a sensing matrix in which continuous glucose monitors (CGMs) detect glucose. This matrix is advantageous for sensing, as other body fluids such as blood and plasma are much more concentrated and have a greater potential to exhibit interference with glucose readings in addition to being more difficult to access, for example, with a semi-permanent device that may be changed 2-3 times per month. Though the ISF is a relatively diluted matrix, there are substances that can introduce an interference to glucose readings. Further, substance interference can compound with other sources of interference, such as interference from equipment. Therefore, interference is a significant problem, especially for dynamic, fast-paced environments such as hospitals, urgent care facilities, emergency rooms, and hospice facilities, which require a high level of accuracy, precision, and consistency.

One existing way to approach substance interference is to provide indications (e.g. labels) to the patient. These indications are often required by law or regulation and may be provided, for example, in a mobile application for monitoring glucose. Problematically, however, such indications are generally static and non-specific to the patient. The indications are typically limited to disclosing that certain predetermined substances, in certain predetermined amounts, are known to interfere with glucose readings. They do little to address the impact of interference. Further, the fact that no interference indication is required for a given substance does not necessarily mean that there is no possibility of interference. For example, variations in the timing or amount of administration, and/or the substance's combination with other substances, can substantially impact glucose readings.

The above problems are exacerbated in the aforementioned dynamic and fast-paced environments. In these settings, different substances, combinations of substances, substance amounts and/or times of administration may be used as compared to an ambulatory setting, thus resulting in a wider array of potential interference sources. Furthermore, since the aforementioned environments are characterized by complicated health situations and nuanced treatment decisions, problems related to accuracy, precision, or consistency of glucose readings are particularly acute. Thus, the static and non-specific nature of typical interference indications leave an informational gap in diabetes patient care.

Accordingly, the present disclosure describes examples of an interference detection and response (IDR) system that can result in higher accuracy and improved treatment decisions for a patient utilizing (e.g., wearing) an analyte sensor system (e.g., a CGM), in both non-ambulatory settings (e.g., fast-paced or dynamic environments such as hospitals, urgent care facilities, emergency rooms, hospice facilities, etc.) and ambulatory settings (e.g., a patient self-care environment). The IDR system can determine whether a substance administered to a patient (or to be administered to a patient) is an interferent for the analyte sensor system by comparing the substance to stored information such as a database of known interferents to determine whether a match exists (indicating that the substance is a known interferent). In certain aspects, it may be determined whether a combination of multiple substances to be administered/already administered to a patient constitutes an interferent for the analyte sensor system (where each of the multiple substances alone/not in combination do not constitute an interferent). This may be done by comparing the combination of substances to stored information such as a database of known interferents to determine whether a match exists (indicating that the combination of substances is a known interferent). This may also be done by predicting a resulting substance that is created when the combination of substances is administered to the patient, and comparing the resulting substance to stored information such as a database of known interferents to determine whether a match exists (indicating that the resulting substance is a known interferent).

In certain aspects, in response to an identification of the substance as an interferent, further interference analysis can be triggered. For example, based on information related to the substance (e.g., pharmacokinetic, biochemical, electrophysiological, and/or other characteristics of the substance), information related to the administration of the substance (e.g., dose and time of administration), patient characteristics (e.g., age, gender, weight, disease history, ethnicity, and/or an Absorption, Distribution, Metabolism, Execration (ADME) model), and/or other information (e.g., historical information for additional patients deemed to share one or more characteristics with the patient), the IDR system can determine an interference effect of the substance on the analyte sensor system. The interference effect can be, for example, an interference bias reflected in analyte measurements, an interference duration, or the like.

In certain aspects, the IDR system can proactively respond or react in real-time to the determined interference effect. For example, the IDR system can compensate for the effect in the analyte measurements (e.g., automatically adjust the measurements), configure reporting or alerting (e.g., blind or block reporting of glucose values or suppress threshold-based alerting), report or alert differently based on different interference effects, glucose levels or amount of data available, or take other proactive actionable steps. In addition, or alternatively, the IDR system can compensate for the effect by removing interference using an anti-fouling approach as described in U.S. Provisional Application No. 63/542,631 and International Application No. PCT/US2024/048484. U.S. Provisional Application No. 63/542,631 and International Application No. PCT/US2024/048484 are hereby incorporated by reference. Although examples are periodically provided herein relative to CGMs, it should be appreciated that the principles described herein are also applicable to other types of analyte monitoring systems configured to monitor analytes such as lactate, potassium, creatinine, etc. Similarly, although certain examples below are described in relation to a diabetic patient, the aspects herein are likewise applicable and useful for detecting interference with analyte sensor systems in connection with any disease or condition for any type of patient.

In this way, an interference effect that one or more substances have on analyte measurements may be eliminated via compensation or preemptive actions. This may improve an accuracy/quality of these analyte measurements and may also maximize an effectiveness of recommendations and/or treatment made in response to an analysis of such analyte measurements. This optimized treatment and/or optimized recommendations may improve a health a patient receiving such treatment/recommendations.

Likewise, if the effects of such interference cannot be removed, alerting one or more users, the patient, etc. to such interference and/or removing/blinding the affected analyte measurements may avoid inaccurate recommendations and/or treatment made in response to an analysis of such analyte measurements.

Also, systems that perform analyte measurement prediction/forecasting may utilize historical analyte measurements for a patient to predict/forecast future analyte measurement for that patient. By eliminating effects of interference that one or more substances may have had on historical analyte measurements, the prediction/forecasting performed based on these historical measurements may be improved. This may in turn improve an accuracy/performance of the systems performing such analyte measurement prediction/forecasting.

Further, it should be noted that, as shown herein, an interference effect that one or more substances have on analyte measurements may be determined in real-time (or nearly real-time). Manually determining this interference effect in real-time or nearly real-time is impossible given the time constraints and complexity of calculations being performed.

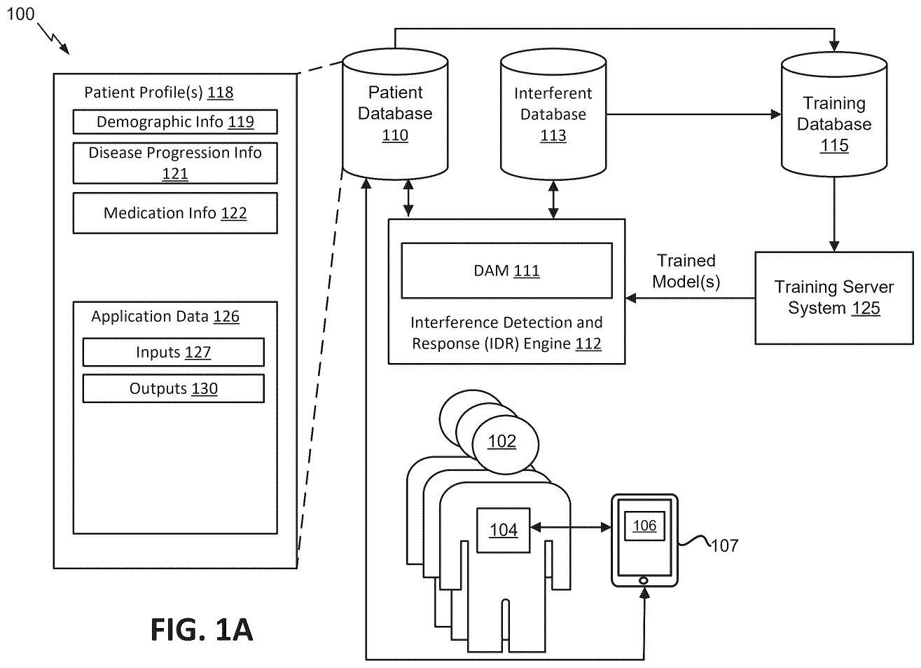

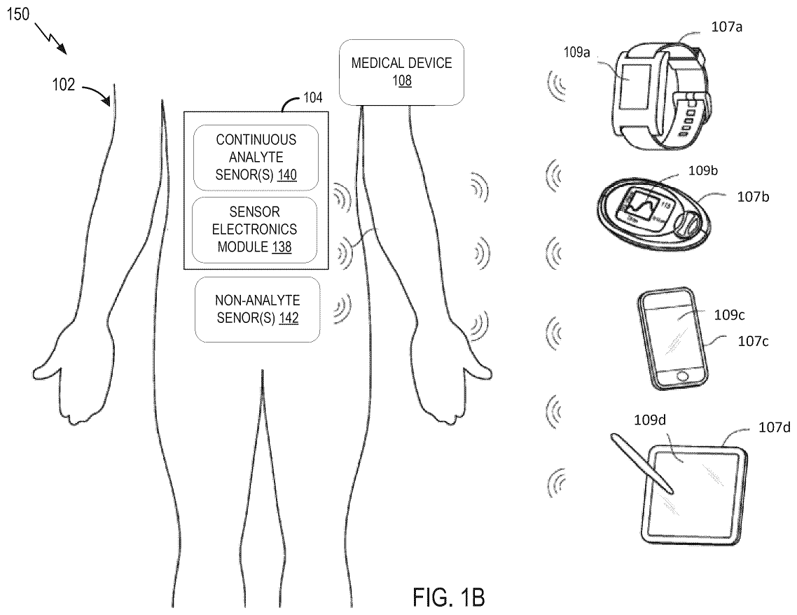

FIG. 1A illustrates an example of a health monitoring system 100, in accordance with certain aspects of the disclosure. The health monitoring system 100 can be utilized for monitoring patient health and displaying data using various user interfaces to users associated with system 100. Each user of system 100, such as patient 102, can interact with a mobile health application, such as mobile health application (“application”) 106 (e.g., a diabetes intervention application that provides decision support guidance), and/or a health monitoring device, such as an analyte sensor system 104 (e.g., a glucose monitoring system). For simplicity, patient 102 is illustrated as a user of system 100. However, it should be appreciated that, in addition, or alternatively, a caregiver of the patient 102, a healthcare professional (HCP), and/or others associated with the patient 102 can be users of system 100. As shown, system 100 can include an analyte sensor system 104, a display device 107 that executes application 106, an IDR engine 112, a patient database 110, an interferent database 113, a training database 115, and a training server system 125.

Analyte sensor system 104 can be configured to generate analyte measurements (also referred to herein as “sensor data” or “analyte data”), for the patient 102, e.g., on a continuous basis, and transmit the analyte measurements to the display device 107 for use by application 106. In some aspects, the analyte sensor system 104 can transmit the analyte measurements to the display device 107 through a wireless connection (e.g., Bluetooth connection). In certain aspects, display device 107 is a smart phone. However, in certain aspects, display device 107 can instead be any other type of computing device such as a laptop computer, a smartwatch, a tablet, or any other computing device capable of executing application 106.

Note that, while in certain examples the analyte sensor system 104 is assumed to be a glucose monitoring system, analyte sensor system 104 can operate to monitor one or more additional or alternative analytes. As discussed, the term “analyte” as used herein is a broad term, and is to be given its ordinary and customary meaning to a person of ordinary skill in the art (and is not to be limited to a special or customized meaning), and refers without limitation to a substance or chemical constituent in the body or a biological sample (e.g., bodily fluids, including, blood, serum, plasma, interstitial fluid, cerebral spinal fluid, lymph fluid, ocular fluid, saliva, oral fluid, urine, excretions, or exudates).

Analytes can include naturally occurring substances, artificial substances, metabolites, and/or reaction products. In some aspects, the analyte measured and used by the devices and methods described herein can include albumin, alkaline phosphatase, alanine transaminase, aspartate aminotransferase, bilirubin, blood urea nitrogen, calcium, CO2, chloride, creatinine, glucose, gamma-glutamyl transpeptidase, hematocrit, lactate, lactate dehydrogenase, magnesium, oxygen, pH, phosphorus, potassium, ketones, sodium, total protein, uric acid, metabolic markers, and/or drugs.

Other analytes are contemplated as well, including but not limited to acetaminophen, dopamine, ephedrine, terbutaline, ascorbate, uric acid, oxygen, d-amino acid oxidase, plasma amine oxidase, xanthine oxidase, NADPH oxidase, alcohol oxidase, alcohol dehydrogenase, pyruvate dehydrogenase, diols, Ros, NO, bilirubin, cholesterol, triglycerides, gentisic acid, ibuprophen, L-Dopa, methyl dopa, salicylates, tetracycline, tolazamide, tolbutamide, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain aspects.

The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), histamine, Advanced Glycation End Products (AGEs) and 5-hydroxyindoleacetic acid (FHIAA).

Application 106 can be a mobile health application that is configured to receive and analyze analyte measurements from the analyte sensor system 104. In some aspects, application 106 can transmit analyte measurements received from the analyte sensor system 104 to a patient database 110 (and/or the IDR engine 112), and the patient database 110 (and/or the IDR engine 112) can store the analyte measurements in a patient profile 118 of patient 102 for processing and analysis as well as for use by the IDR engine 112 to detect and respond to substance interference. In various aspects, application 106 can provide data processed or generated by the IDR engine 112 to the patient 102 using user interfaces via the application 106. In some aspects, application 106 can store the analyte measurements in a patient profile 118 of patient 102 locally for processing and analysis as well as for use by the IDR engine 112 to dynamically detect and handle substance interference.

Interferent database 113 can include, for example, a list of medications that are known to interfere with various analyte sensor systems at issue, such as the analyte sensor system 104, and/or that are known to pose a risk of such interference. In some aspects, the interferent database 113 can include, for example, a list of medications with known or predicted risk based on, for example, their biochemical or electrophysiological characteristics. In some cases, the interferent database 113 can be based on a design of the analyte sensor system 104 as well as in-vitro and/or in-vivo evidence of interference. For example, it may be known from field complaints, and/or through substance interference testing, that N-Hydroxyurea and acetaminophen interfere with the analyte sensor system 104, thus meriting their inclusion in the interferent database 113. In various aspects, substances identified through methods such as the foregoing, and/or substances with similar chemical characteristics to such substances, may be included in the interferent database 113.

The IDR engine 112 can predictively and/or proactively detect and dynamically handle substance interference with the analyte sensor system 104. In certain aspects, the IDR engine 112 can detect an administration of a substance (e.g., an oral, intravenous, parenteral or other type of administration of a medication), or combination of substances, to the patient 102, for example, from stored data in the patient database 110, other stored data, and/or user entry (e.g., by the patient 102 or an HCP). In some cases, the administration can be a past or completed administration that is detected, for example, from stored information related to the patient 102 such as an electronic medical record (EMR) or the like. In other cases, the administration can be a planned and/or incomplete administration that is detected, for example, from stored information related to the patient 102 such as a prescription, a patient care plan, a medication administration schedule, or the like. Although the IDR engine 112 can operate with respect to a combination of substances, for simplicity of description, its operation will be described relative to a single substance.

In certain aspects, the IDR engine 112 can identify the substance as an interferent, or as a non-interferent, based on stored information in the interferent database 113. For example, information related to the administration of the substance (e.g., substance name or other identifier, dose, and/or time of administration) can be searched against the interferent database 113. If the search yields a match, the substance can be identified as an interferent. Otherwise, if the search does not yield a match, the substance can be identified, or treated, as a non-interferent such that no further action is taken based thereon.

In certain aspects, the IDR engine 112 can trigger further interference analysis related to the substance in cases where the substance is identified as an interferent. For example, the IDR engine 112 can generate an interference effect of the substance on the analyte sensor system 104. In general, the interference effect quantifies or otherwise characterizes an impact of the substance on analyte measurements produced by the analyte sensor system 104. The interference effect can based on, for example, information related to the substance (e.g., pharmacokinetic, biochemical, electrophysiological, and/or other characteristics of the substance), the information related to the substance's administration, patient characteristics (e.g., age, gender, weight, disease history, ethnicity and/or an ADME model), and/or other information (e.g., historical information for additional patients deemed to share one or more characteristics with the patient). Blood glucose data may also be determined and used to confirm an accuracy of a predicted interference effect and/or confirm an amount of bias determined to address such interference effect.

In certain aspects, the IDR engine 112 can determine and execute an interference response based on the interference effect. The interference response can include, for example, one or more defined actions based on the interference effect. For example, the IDR engine 112 can configure reporting or alerting, for example, by blinding or blocking reporting (e.g., blocking analyte measurements from presentation to the patient 102), suppressing alerting, and/or reporting or alerting differently based on different interference effects, analyte measurements or amount of data available. In another example, the IDR engine 112 can cause an interference notification to be generated and presented that informs the patient 102, for example, that the analyte measurements may be less accurate for a period of time (e.g., the interference duration) due to the administration of the substance. In some cases, the interference notification can provide information related to the interference effect, such as an interference bias and/or an interference duration.

In some aspects, the interference notification may include a preemptive alert that is provided to one or more users. For example, if a plan to administer a dose of a particular substance to a user is identified (e.g., via a drug administration order within a hospital environment, via a scheduled calendar entry for a user, etc.), the substance and dose may be automatically analyzed prior to the administration of the substance. If it is determined that the planned dose will result in an interference effect for one or more analyte measurements produced by the analyte sensor system 104, this interference may be presented to one or more users (e.g., patient, doctor, etc.).

In some aspects, one or more alternative substances (and appropriate doses corresponding to the dose of the interferent substance) may be identified (e.g., by referencing a database containing a list of known interferent substances and predetermined alternative substances for those interferent substances that do not result in an interference effect for the one or more analyte measurements). The alternative substances and doses may be presented to one or more users.

In some aspects, the interference response determined by the IDR engine 112 can involve compensating for the interference. For example, the IDR engine 112 can initiate algorithmic compensation for the interference effect. The algorithmic compensation can include automatically adjusting the analyte measurements and presenting the adjusted analyte measurements to the patient 102. In some cases, the adjusted analyte measurements may be presented in place of the unadjusted analyte measurements generated by the analyte sensor system 104, while in other cases the adjusted analyte measurements may be presented in conjunction with the unadjusted measurements.

In some aspects, a confidence value may be calculated for the algorithmic compensation being performed. The calculated confidence value may be compared to a threshold confidence value, and in response to determining that the calculated confidence value does not meet or exceed the threshold confidence value, the adjusted analyte measurements may be discarded, may be shown with a disclaimer (e.g., that such measurements are not to be relied upon, etc.), etc. One or more other methods for obtaining analyte readings that are not affected by the interferent substance(s) may also be identified (e.g., by querying a database storing analyte reading methods and associated interferents for such methods), and a suggestion to use one or more of these methods (such as a blood glucose monitor (BGM), fingerstick blood glucose test, etc.) may be presented to one or more users.

Also, in response to determining that the planned dose will result in an interference effect for one or more analyte measurements produced by the analyte sensor system 104, a user (such as a doctor, caregiver, etc.) may be asked to confirm (e.g., via a GUI of the IDR engine 112) that the dose has been administered despite the anticipated interference. One or more additional users (e.g., a supervisor, etc.) may be asked to confirm (e.g., via a GUI of the IDR engine 112) the administration of the dose, as well as the dose amount and substance, to ensure the accuracy of interference compensation being performed.

In some aspects, the presentation of the adjusted analyte measurements can be accompanied by, or prefaced with, an interference alert or disclaimer to the effect that analyte measurements are being algorithmically adjusted to compensate for the interference. In addition, or alternatively, the adjusted analyte measurements can be presented in a visually different way than the unadjusted analyte measurements generated by the analyte sensor system for emphasis (e.g., different color, font, location, etc.). In addition, or alternatively, by way of further example, the IDR engine 112 can compensate for the interference effect by removing interference using an anti-fouling approach as described in U.S. Provisional Application No. 63/542,631 and International Application No. PCT/US2024/048484.

In various aspects, the adjusted analyte measurements may be presented to one or more users (e.g., utilizing a graphical user interface (GUI), may be provided to one or more third-party applications, etc. The original analyte measurements (prior to adjustment) may also be presented/provided. For example, the original analyte measurements may be presented on a graph of a GUI utilizing a first color, pattern, etc., and the adjusted analyte measurements may be presented on the same or different graph of the GUI utilizing a second color, pattern, etc. that is different from the first color, pattern, etc.

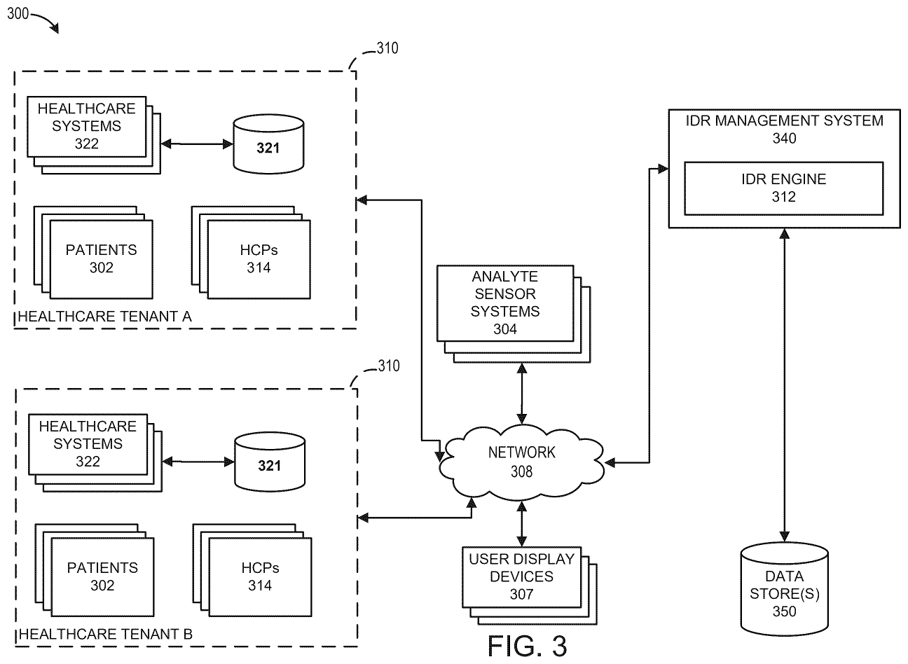

In various aspects, compensating for interference, for example, in any of the ways described above, technically improves sensor systems such as the analyte sensor system 104. As noted above, such analyte sensor systems may sometimes generate inaccurate and/or unreliable measurements due to interference, which measurements might otherwise be used as a basis for reporting or alerting. Improving such sensor systems (and/or systems that receive data from such systems) to address interference is a technical improvement, as it results in greater accuracy and reliability and more informed treatment decisions. In various aspects, the IDR engine 112 can be adapted to an ambulatory setting (e.g., a patient self-care environment) and/or a non-ambulatory setting (e.g., fast-paced or dynamic environments such as hospitals, urgent care facilities, emergency rooms, hospice facilities, etc.) to predictively and/or proactively detect and dynamically handle substance interference with analyte sensor systems. The example of FIG. 1A can correspond to an example of an ambulatory setting. Example operability of the IDR engine 112 in a non-ambulatory setting will be described relative to FIG. 3.

In certain aspects, IDR engine 112 can be implemented as a set of software instructions with one or more software modules, including a data analysis module (DAM) 111. In some aspects, IDR engine 112 executes entirely on one or more computing devices in a private or a public cloud. In some other aspects, IDR engine 112 executes partially on one or more local devices, such as display device 107 (e.g., via application 106) and/or analyte sensor system 104, and partially on one or more computing devices in a private or a public cloud. In some other aspects, IDR engine 112 executes entirely on one or more local devices, such as display device 107 (e.g., via application 106) and/or analyte sensor system 104.

In various aspects, DAM 111 of IDR engine 112 can be configured to process and/or generate data for the IDR engine 112. In certain aspects, DAM 111 of IDR engine 112 can be configured to receive and/or process a set of inputs 127 (described in more detail below) (also referred to herein as “input data”) to determine one or more metrics 130. Inputs 127 can be stored in the patient profile 118 in the patient database 110. DAM 111 can fetch inputs 127 from the patient database 110 and compute a plurality of metrics 130 which can then be stored as application data 126 in the patient profile 118. Such metrics 130 can include health-related metrics.

In certain aspects, application 106 is configured to take, as input, information relating to patient 102, and to store the information in a patient profile 118 for patient 102 in patient database 110. The patient profile 118 can include patient characteristics of the type described previously. For example, application 106 can obtain and record patient 102's demographic info 119, disease progression info 121, and/or medication info 122 in patient profile 118. In certain aspects, demographic info 119 can include one or more of the patient 102's age, body mass index (BMI), ethnicity, gender, etc.

In certain aspects, disease progression info 121 can include information about the patient 102's disease, such as, for diabetes, whether the patient is Type I, Type II, pre-diabetes, or whether the patient has gestational diabetes. In certain aspects, disease progression info 121 also includes the length of time since diagnosis, the level of disease control, level of compliance with disease management therapy, predicted pancreatic function, other types of diagnosis (e.g., heart disease, obesity) or measures of health (e.g., heart rate, exercise, stress, sleep, etc.), and/or the like. In certain aspects, medication info 122 can include information about the amount and type of medication taken by patient 102, such as insulin or non-insulin diabetes medications and/or non-diabetes medication taken by patient 102.

In certain aspects, application 106 can obtain demographic info 119, disease progression info 121, and/or medication info 122 from the patient 102 in the form of user input or from other sources. In certain aspects, as some of this information changes, application 106 can receive updates from the patient 102 or from other sources. In certain aspects, patient profile 118 associated with the patient 102, as well as other patient profiles associated with other patients are stored in a patient database 110, which is accessible to application 106, as well as to the IDR engine 112, over one or more networks (not shown).

In certain aspects, application 106 collects inputs 127 through patient 102 input, other user (e.g., HCP 314) input and/or a plurality of other sources, including analyte sensor system 104, other applications running on display device 107, one or more healthcare systems 322, and/or one or more other sensors and devices. In certain aspects, such sensors and devices include one or more of, but are not limited to, an insulin pump, other types of analyte sensors, sensors or devices provided by display device 107 (e.g., accelerometer, camera, global positioning system (GPS), heart rate monitor, etc.) or other user accessories (e.g., a smartwatch), or any other sensors or devices that provide relevant information about the patient 102. In certain aspects, patient profile 118 also stores application configuration information indicating the current configuration of application 106, including its features and settings.

Patient database 110, in some aspects, refers to a storage server that can operate in a public or private cloud. Patient database 110 can be implemented as any type of data store, such as relational databases, non-relational databases, key-value data stores, file systems including hierarchical file systems, and the like. In some exemplary implementations, patient database 110 is distributed. For example, patient database 110 can comprise a plurality of persistent storage devices, which are distributed. Furthermore, patient database 110 can be replicated so that the storage devices are geographically dispersed.

Patient database 110 can include other patient profiles 118 associated with a plurality of other patients served by system 100. More particularly, similar to the operations performed with respect to the patient 102, the operations performed with respect to these other patients can utilize an analyte monitoring system, such as analyte sensor system 104, and also interact with the same application 106, copies of which execute on the respective display devices of the other patients 102. For such patients, patient profiles 118 are similarly created and stored in patient database 110.

In certain aspects, IDR engine 112 can utilize one or more trained machine learning models. In the illustrated aspect of FIG. 1, IDR engine 112 can utilize trained machine learning model(s) provided by a training server system 125. Although depicted as a separate server for conceptual clarity, in certain aspects, training server system 125 and IDR engine 112 can operate as a single server or system. That is, the model can be trained and used by a single server, or can be trained by one or more servers and deployed for use on one or more other servers or systems. In certain aspects, the model can be trained on one or many virtual machines (VMs) running, at least partially, on one or many physical services in relational and or non-relational database formats.

Training server system 125 is configured to train the machine learning model(s) using training data, which can include data (e.g., from patient profiles) associated one or more patients (e.g., users or non-users of analyte sensor system 104 and/or application 106) and/or data associated with one or more substances (e.g., from interferent database 113). The training data can be stored in training database 115 and can be accessible to training server system 125 over one or more networks (not shown) for training the machine learning model(s).

The training data refers to a dataset that has been featurized and labeled. For example, the dataset can include a plurality of data records, each including information corresponding to an administration of a substance to patient (e.g., associated with a different patient profile stored in patient database 110), where each data record is featurized and labeled. In machine learning and pattern recognition, a feature is an individual measurable property or characteristic. Generally, the features that best characterize the patterns in the data are selected to create predictive machine learning models. Data labeling is the process of adding one or more meaningful and informative labels to provide context to the data for learning by the machine learning model.

As an illustrative example, each relevant characteristic of a patient, which is reflected in a corresponding data record, can be a feature used in training the machine learning model. Such features can include demographic information (e.g., age, gender, ethnicity, etc.), analyte information (e.g., glucose metrics, such as a glucose baseline, minimum and maximum daily glucose levels, glucose peak following meals, drinks, or food, glucose clearance rate following glucose peak, and/or glucose levels during and after exercise, etc.), non-analyte sensor information (e.g., heart rate, temperature, etc.), diabetes information (e.g., diabetes diagnosis, insulin resistance), comorbidities (e.g., hyperglycemia, hypoglycemia, kidney conditions and diseases, hypertension, etc.), substance information (e.g., pharmacokinetic, biochemical, electrophysiological, and/or other characteristics of the substance), substance administration information (e.g., substance name or other identifier, dose, and/or time of administration), and/or any other information relevant to detecting interferents, determining interference effects, determining interference responses, executing interference responses (e.g., compensating for interference) and/or the like.

In addition, the data record is labeled with information the corresponding model is being trained to predict. In one example, if a model is being trained to determine, for particular patients and substances, an interference effect, then the data records in the training dataset are labeled with such effect (e.g., interference bias and/or duration). In another example, if a model is being trained to output an interference response, then the data records in the training dataset are labeled with one or more of predictions of such response.

The model(s) are then trained by training server system 125 using the featurized and labeled training data. In particular, the features of each data record can be used as input into the machine learning model(s), and the generated output can be compared to label(s) associated with the corresponding data record. The model(s) can compute a loss based on the difference between the generated output and the provided label(s). This loss is then used to modify the internal parameters or weights of the model. By iteratively processing each data record corresponding to each historical patient, the model(s) can be iteratively refined to generate accurate predictions of interferent classification, interferent effect, interferent response, etc.

As illustrated in FIG. 1, training server system 125 deploys these trained model(s) to IDR engine 112 for use during runtime. For example, IDR engine 112 can obtain patient profile 118 associated with a patient and stored in patient database 110, use information in patient profile 118 as input into the trained model(s), and output a prediction indicative of whether a substance that has been administered, or is planned to be administered, is an interferent. In addition, or alternatively, the IDR engine 112 can output an interference effect of such substance and/or an interference response to such interference (e.g., whether to compensate and/or how to compensate).

In certain aspects, a patient's own historical data can be used by training server system 125 to train a personalized model for the patient that provides interference detection and response. For example, in certain aspects, a model trained based on population data can be initially used for interference detection and response. However, after collecting personalized information (e.g., analyte sensor information, non-analyte sensor information, disease state, etc.) associated with the patient, the personalized information can be used for further personalizing the model. For example, information obtained over time from the patient can be used to more accurately determine interference effect of particular substances and compensate for such interference (e.g., by adjusting measurements more accurately).

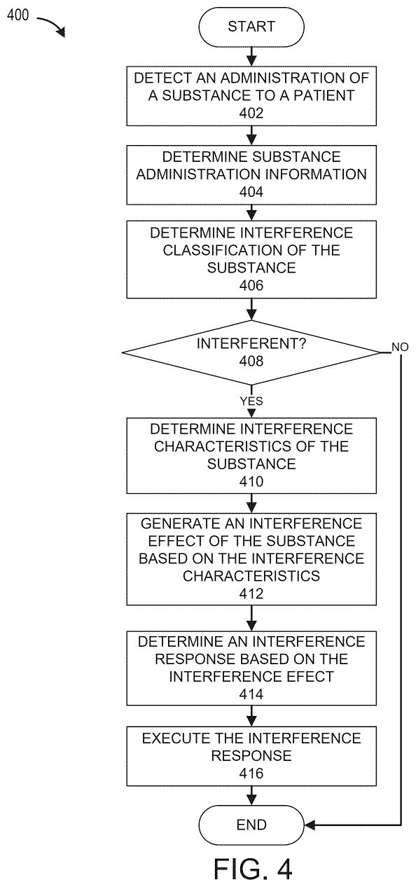

In certain other aspects, rules-based models can be used. For example, a rules-based model can be used to map a given administration of a substance to a patient to an interferent classification (e.g., interferent or non-interferent), an interference effect (e.g., interference bias and/or duration), an interference response (e.g., alerting or compensation), a manner of executing the interference response (e.g., how to compensate, and/or the like. Some example rules are discussed herein in relation to block 412 of FIG. 4.

FIG. 1B is a diagram 150 illustrating an example of the analyte sensor system 104 including an example continuous analyte sensor(s) with sensor electronics, in accordance with certain aspects of the present disclosure. For example, the analyte sensor system 104 can be configured to continuously monitor one or more analytes of a patient, in accordance with certain aspects of the present disclosure.

As shown in FIG. 1B, the analyte sensor system 104 in the illustrated aspect includes a sensor electronics module 138 and one or more continuous analyte sensor(s) 140 (individually referred to herein as the continuous analyte sensor 140 and collectively referred to herein as the continuous analyte sensors 140) associated with a sensor electronics module 138. The sensor electronics module 138 can be in wireless communication (e.g., directly or indirectly) with one or more display devices 107a, 107b, 107c, and 107d. In certain aspects, the sensor electronics module 138 can also be in wireless communication (e.g., directly or indirectly) with one or more medical devices 108 (individually referred to herein as the medical device 108 and collectively referred to herein as the medical devices 108).

In certain aspects, a continuous analyte sensor 140 can comprise a sensor for detecting and/or measuring analyte(s). The continuous analyte sensor 140 can be a multi-analyte sensor configured to continuously measure two or more analytes (e.g., ketone, glucose) or a single analyte sensor configured to continuously measure a single analyte as a non-invasive device, a subcutaneous device, a transcutaneous device, a transdermal device, and/or an intravascular device.

In certain aspects, the continuous analyte sensor 140 can be configured to continuously measure analyte levels of a patient using one or more measurement techniques, such as enzymatic, chemical, physical, electrochemical, spectrophotometric, polarimetric, calorimetric, iontophoretic, radiometric, immunochemical, and the like. In certain aspects, the continuous analyte sensor 140 provides a data stream indicative of the concentration of one or more analytes in the patient. The data stream can include raw data signals which can be converted into a calibrated and/or filtered data stream used to provide estimated analyte value(s) to the patient.

In certain aspects, the continuous analyte sensor 140 can be a multi-analyte sensor, configured to continuously measure multiple analytes in a patient's body. For example, in certain aspects, the continuous multi-analyte sensor 140 can be a single sensor configured to measure glucose, ketones, and/or other blood analytes in the patient's body.

In certain aspects, one or more multi-analyte sensors can be used in combination with one or more single analyte sensors. As an illustrative example, a multi-analyte sensor can be configured to continuously measure ketone and glucose and can, in some cases, be used in combination with one or more other analyte sensors configured to measure only, for example, hydration levels or protein levels.

In certain aspects, the sensor electronics module 138 includes electronic circuitry associated with measuring and processing the continuous analyte sensor data, including prospective algorithms associated with processing and calibration of the sensor data. The sensor electronics module 138 can be physically connected to the continuous analyte sensor(s) 140 and can be integral with (non-releasably attached to) or releasably attachable to the continuous analyte sensor(s) 140. The sensor electronics module 138 can include hardware, firmware, and/or software that enables measurement of levels of analyte(s) via a continuous analyte sensor(s) 140. For example, the sensor electronics module 138 can include a potentiostat, a power source for providing power to the sensor, other components useful for signal processing and data storage, and a telemetry module for transmitting data from the sensor electronics module to one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

In some aspects, the display devices 107a, 107b, 107c, and/or 107d are configured for displaying displayable sensor data, including analyte data, which can be transmitted by the sensor electronics module 138. In addition, or alternatively, the display devices 107a, 107b, 107c, and/or 107d are configured for displaying reports, notifications and/or alerts as described herein, which data can be generated by the IDR engine 112. Each of the display devices 107a, 107b, 107c, or 107d can include a display such as a touchscreen display 109a, 109b, 109c, /or 109d for displaying sensor data to a user and/or receiving inputs from the user. For example, a graphical user interface can be presented to the user for such purposes. In some aspects, the display devices 107a, 107b, 107c, and 107d can include other types of user interfaces such as a voice user interface instead of, or in addition to, a touchscreen display for communicating sensor data to the user of the display device and/or receiving user inputs. The display devices 107a, 107b, 107c, and 107d can be examples of the display device 107 illustrated in FIG. 1A used to display sensor data to the patient 102 and/or receive input from the patient 102.

In some aspects, one, some, or all of the display devices are configured to display or otherwise communicate the sensor data as it is communicated from the sensor electronics module (e.g., in a data package that is transmitted to respective display devices), without any additional prospective processing required for calibration and real-time display of the sensor data.

The plurality of display devices can include a custom display device specially designed for displaying certain types of displayable sensor data associated with analyte data received from sensor electronics module and/or interference-related data. In certain aspects, the plurality of display devices can be configured for providing alerts/alarms based on the displayable sensor data. The display device 107b is an example of such a custom device. In some aspects, one of the plurality of display devices is a smartphone, such as the display device 107c which represents a mobile phone, using a commercially available operating system (OS), and configured to display a graphical representation of the continuous sensor data (e.g., including current and historic data) and/or interference-related data. Other display devices can include other hand-held devices, such as the display device 107d which represents a tablet, the display device 107a which represents a smartwatch, the medical device 108 (e.g., an insulin delivery device or a blood glucose meter), and/or a desktop or laptop computer (not shown). Display device 107d and display device 107a can similarly be configured to display graphical representations of the continuous sensor data (e.g., including current and historic data) and/or interference-related data.

Because different display devices provide different user interfaces, content of the data packages (e.g., amount, format, and/or type of data to be displayed, alarms, and the like) can be customized (e.g., programmed differently by the manufacture and/or by an end user) for each particular display device. Accordingly, in certain aspects, a plurality of different display devices can be in direct wireless communication with a sensor electronics module (e.g., such as an on-skin sensor electronics module 138 that is physically connected to continuous analyte sensor(s) 140) during a sensor session to enable a plurality of different types and/or levels of display and/or functionality associated with the displayable sensor data. In certain aspects, the type of alarms customized for each particular display device, the number of alarms customized for each particular display device, the timing of alarms customized for each particular display device, and/or the threshold levels configured for each of the alarms (e.g., for triggering) are based on the current health of a patient, the state of a patient's analyte levels, current treatment recommended to a patient, and/or physiological parameters of a patient.

As mentioned, the sensor electronics module 138 can be in communication with a medical device 108. The medical device 108 can be a passive device in some example aspects of the disclosure. For example, the medical device 108 can be an insulin pump for administering insulin to a patient.

In certain aspects, one or more other non-analyte sensors 142 (individually referred to herein as the non-analyte sensor 142 and collectively referred to herein as the non-analyte sensor 142) can be in communication with any of the display devices 107. The non-analyte sensors 142 can include, but are not limited to, an altimeter sensor, an accelerometer sensor, a temperature sensor, a respiration rate sensor, a sweat sensor, etc. The non-analyte sensors 142 can also include monitors such as heart rate monitors, ECG monitors, blood pressure monitors, pulse oximeters, or the like.

The non-analyte sensors 142 can also include data systems for measuring non-patient specific phenomena such as time, ambient pressure, or ambient temperature which could include an atmospheric pressure sensor, an external air temperature sensor or a clock, timer. In some aspects, the non-analyte sensors 142 can be, or include, an activity monitor, for example, that includes a combination of the foregoing sensors, such as an accelerometer sensor, a heart rate monitor, GPS sensor, and/or the like. In addition, or alternatively, the non-analyte sensors 142, such as an activity monitor, can be, or be integrated in, one or more of the display devices 107 such as, for example, the display device 107a which represents a smartwatch. One or more of these non-analyte sensors 142 can provide data to the IDR engine 112.

In certain aspects, a wireless access point (WAP) can be used to couple one or more of the analyte sensor system 104, the plurality of display devices, the medical device(s) 108, and/or the non-analyte sensor(s) 142 to one another. For example, the WAP can provide Wi-Fi and/or cellular connectivity among these devices. Near Field Communication (NFC) and/or Bluetooth can also be used among devices depicted in the diagram 150 of FIG. 1B.

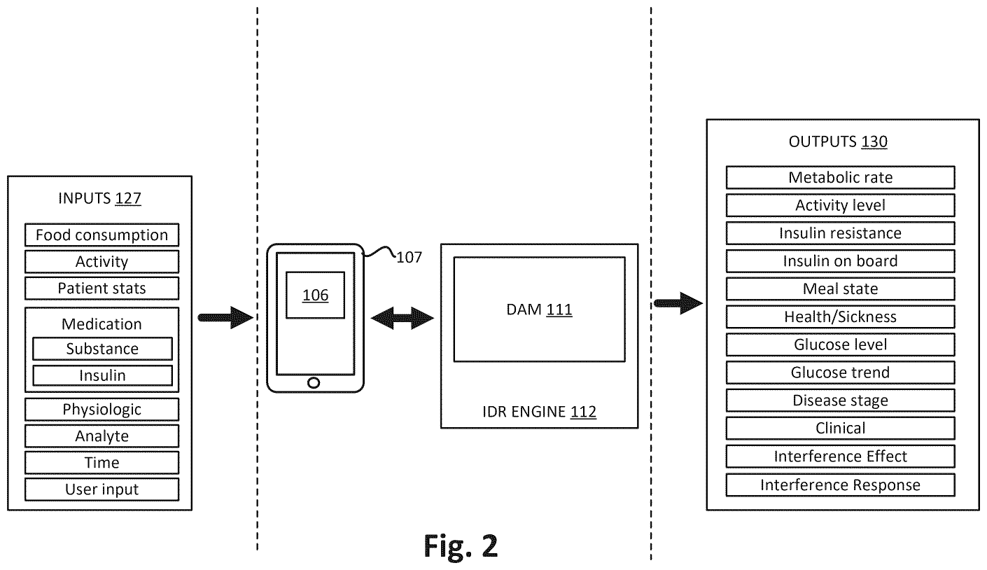

FIG. 2 illustrates example inputs and example metrics that are generated based on the inputs in accordance with certain aspects of the disclosure. In particular, FIG. 2 illustrates example inputs 127 on the left, application 106 and IDR engine 112, with DAM 111, in the middle, and example metrics 130 on the right. In certain aspects, application 106 can obtain inputs 127, in the form of time-series data, through one or more channels (e.g., continuous analyte sensor(s) 140, non-analyte sensor(s) 142, various applications executing on display device 107, etc.). Inputs 127 can be further processed by DAM 111 to output a plurality of metrics, such as metrics 130. Further, inputs (e.g., inputs 127) and metrics (e.g., metrics 130) can be used by the DAM 111 and/or any computing device in the system 100 to perform various processes in detecting and dynamically handling substance. Any of inputs 127 can be used for computing any of metrics 130. In certain aspects, each one of metrics 130 can correspond to one or more values, e.g., discrete numerical values, ranges, or qualitative values (high/medium/low or stable/unstable). In some aspects, some or all of metrics 130 can include time-series data and/or be provided in the form of time-series data.

In certain aspects, inputs 127 include food consumption information. Food consumption information can include information about one or more of meals, snacks, and/or beverages, such as one or more of the size, content (carbohydrate, fat, protein, etc.), sequence of consumption, and time of consumption. In certain aspects, food consumption can be provided by the user through manual entry, by providing a photograph through an application that is configured to recognize food types and quantities, and/or by scanning a bar code or menu. In various examples, meal size can be manually entered as one or more of calories, quantity (e.g., ‘three cookies’), menu items (e.g., ‘Royale with Cheese’), and/or food exchanges (1 fruit, 1 dairy). In some examples, meals can also be entered with the user's typical items or combinations for this time or context (e.g., workday breakfast at home, weekend brunch at restaurant). In some examples, meal information can be received via a convenient user interface provided by application 106.

In certain aspects, inputs 127 include activity information. Activity information can be provided, for example, by the one or more non-analyte sensors 142 of FIG. 1B (e.g., by an accelerometer sensor on a wearable device such as a watch, fitness tracker, and/or patch). In certain aspects, activity information can additionally be provided through manual input by patient 102. Activity information can include exercise related information, sleep information, and other types of information related to the user's activity or lack thereof.

In certain aspects, inputs 127 include patient statistics, such as one or more of age, height, weight, body mass index, body composition (e.g., % body fat), stature, build, or other information. Patient statistics can be provided through a user interface, by interfacing with an electronic source such as an electronic medical record, and/or from measurement devices. The measurement devices can include one or more of a wireless, e.g., Bluetooth-enabled, weight scale and/or camera, which can, for example, communicate with the display device 107 to provide patient data.

In certain aspects, inputs 127 include information relating to the user's substance intake (e.g., medication intake). For example, the user's substance intake can include a substance such as a medication that has been or will be administered to the user. Such substance information can be obtained, for example, from an EMR, a stored prescription, and/or be manually entered. In another example, the user's substance intake can include the user's insulin delivery. Such insulin information can be received, via a wireless connection on a smart pen, via user input, and/or from an insulin pump (e.g., medical device 108). Insulin delivery information can include one or more of insulin volume, time of delivery, etc. Other configurations, such as insulin action time or duration of insulin action, can also be received as inputs.

In certain aspects, inputs 127 include physiological information received from non-analyte sensor(s) 142, which can detect one or more of heart rate, respiration, oxygen saturation, body temperature, etc. (e.g., to detect illness, stress levels, etc.).

In certain aspects, inputs 127 include analyte data, which can be provided as input from analyte sensor system 104, for example, in any of the ways described with respect to FIG. 1A. An example of analyte data is glucose data, which can be provided and/or stored as a time series corresponding to time-stamped glucose measurements over time. Other types of analyte data, such as ketone data, potassium data, lactate data, etc., can similarly be provided and/or stored as a time series. In certain aspects, inputs 127 include time, such as time of day, or time from a real-time clock.

As described above, in certain aspects, DAM 111 determines or computes metrics 130 based on inputs 127 associated with patient 102. An example list of metrics 130 is illustrated in FIG. 2. In certain aspects, metrics 130 determined or computed by DAM 111 include metabolic rate. Metabolic rate is a metric that can indicate or include a basal metabolic rate (e.g., energy consumed at rest) and/or an active metabolism, e.g., energy consumed by activity, such as exercise or exertion. In some examples, basal metabolic rate and active metabolism can be tracked as separate metric. In certain aspects, the metabolic rate can be calculated by DAM 111 based on one or more of inputs 127, such as one or more of activity information, sensor input, time, user input, etc.

In certain aspects, metrics 130 determined or computed by DAM 111 include an activity level metric. The activity level metric can indicate a level of activity of the user. In certain aspects, the activity level metric can be determined, for example, based on input from an activity sensor or other physiologic sensors. In certain aspects, the activity level metric can be calculated by DAM 111 based on one or more of inputs 127, such as one or more of activity information, physiological information, analyte data, time, user input, etc. Activity level can indicate whether the user is exercising, at rest, sleeping, etc.

In certain aspects, metrics 130 determined or computed by DAM 111 include an insulin resistance metric (also referred to herein as an “insulin resistance”). The insulin resistance metric can be determined using historical data, real-time data, or a combination thereof, and can, for example, be based upon one or more inputs 127, such as one or more of food consumption information, blood glucose information, insulin delivery information, the resulting glucose levels, etc. In certain aspects, the insulin on board metric can be determined using insulin delivery information, and/or known or learned (e.g., from patient data) insulin time action profiles, which can account for both basal metabolic rate (e.g., update of insulin to maintain operation of the body) and insulin usage driven by activity or food consumption.

In certain aspects, metrics 130 determined or computed by DAM 111 include a meal state metric. The meal state metric can indicate the state the user is in with respect to food consumption. For example, the meal state can indicate whether the user is in one of a fasting state, pre-meal state, eating state, post-meal response state, or stable state. In certain aspects, the meal state can also indicate nourishment on board, e.g., meals, snacks, or beverages consumed, and can be determined, for example from food consumption information, time of meal information, and/or digestive rate information, which can be correlated to food type, quantity, and/or sequence (e.g., which food/beverage was eaten first.).

In certain aspects, metrics 130 determined or computed by DAM 111 include health and sickness metrics. Health and sickness metrics can be determined, for example, based on one or more of user input (e.g., pregnancy information or known sickness information), from non-analyte sensor(s) 142, such as physiologic sensors (e.g., temperature), activity sensors, or a combination thereof. In certain aspects, based on the values of the health and sickness metrics, for example, the user's state can be defined as being one or more of healthy, ill, rested, or exhausted. In certain aspects, health and sickness metric can indicate the user's heart rate, stress level, etc.

In certain aspects, metrics 130 determined or computed by DAM 111 include analyte level metrics. Analyte level metrics can be determined from analyte data (e.g., glucose measurements obtained from analyte sensor system 104). In some examples, an analyte level metric can also be determined, for example, based upon historical information about analyte levels in particular situations, e.g., given a combination of food consumption, insulin, and/or activity. An analyte level metric can include a rate of change of the analyte, time in range, time spent below a threshold level, time spent above a threshold level, or the like. In certain aspects, an analyte trend can be determined based on the analyte level over a certain period of time. As described above, example analytes can include glucose, ketones, lactate, potassium and others described herein.

In certain aspects, metrics 130 determined or computed by DAM 111 include a disease stage. For example, disease stages for Type II diabetics can include a pre-diabetic stage, an oral treatment stage, and a basal insulin treatment stage. In certain aspects, degree of glycemic control (not shown) can also be determined as an outcome metric, and can be based, for example, on one or more of glucose levels, variation in glucose level, or insulin dosing patterns.

In certain aspects, metrics 130 determined or computed by DAM 111 include clinical metrics. Clinical metrics generally indicate a clinical state a user is in with respect to one or more conditions of the user, such as diabetes. For example, in the case of diabetes, clinical metrics can be determined based on glycemic measurements, including one or more of A1C, trends in A1C, time in range, time spent below a threshold level, time spent above a threshold level, and/or other metrics derived from glucose values. In certain aspects, clinical metrics can also include one or more of estimated A1C, glycemic variability, hypoglycemia, and/or health indicator (time magnitude out of target zone).

In certain aspects, metrics 130 determined or computed by DAM 111 include an interference effect and an interference response related to a substance that has been, or is planned to be, administered to the patient 102. The interference effect can be generated, for example, based on information related to the substance, information related to the administration of the substance, patient characteristics, and/or other information, as described previously. The interference response can be based on the interference effect and can include, for example, one or more defined actions based on the interference effect, such as interference alerting and/or initiating algorithmic compensation for the effect of the substance on the analyte measurements produced by the analyte sensor system 104, as described previously.

FIG. 3 illustrates an example of a computing environment 300 for implementing an IDR management system 340. In various aspects, the IDR management system 340 can enable interference detection and response across the same or multiple healthcare facilities, for example, in non-ambulatory settings. The computing environment 300 includes the IDR management system 340, healthcare tenants 310, analyte sensor systems 304, user display devices 307, and one or more data stores 350, each of which can communicate over a network 308. The network 308 can be, or include, one or more of a private network, a public network, a local or wide area network, a portion of the Internet, combinations of the same, and/or the like. The network 308 can include, for example, interfaces (e.g., application programming interfaces) for enabling interaction and communication between and among the components and systems of the computing environment 300.

In certain aspects, the IDR management system 340 can centrally manage interference detection and response for the healthcare tenants 310, each of which can correspond to a healthcare facility that provides a non-ambulatory setting for patient care, such as a hospital, urgent care facility, emergency room, hospice facility, a system of any of the foregoing and/or the like. In general, the healthcare tenants 310 can each be considered an abstraction of IDR deployments managed by the IDR management system 340 and the systems, data sources and users with which those deployments interact.

For example, one of the healthcare tenants 310 is shown as owned or operated by “Healthcare Tenant A” while another system 458 is owned or operated by a different tenant, “Healthcare Tenant B.” The healthcare tenants 310 shown can be owned or operated by the same or different entities. For example, in some cases, Healthcare Tenants A and B can represent customers (e.g., healthcare entities such as hospitals, hospital systems, and/or healthcare facilities described previously) of an operator of the IDR management system 340. Although the term “tenant” is used herein to describe the healthcare tenants 310 or owners/operators thereof, in addition to having its ordinary meaning, the term “tenant” can, but need not, refer to tenancy in a multitenant software architecture.

The healthcare tenants 310 are each shown to include patients 302, HCPs 314, data sources 321, and healthcare systems 322. For each healthcare tenant of the healthcare tenants 310, the patients 302 can correspond to patients (e.g., admitted patients) of that healthcare tenant. Similarly, for each healthcare tenant of the healthcare tenants 310, the HCPs 314 can correspond to HCPs, such as doctors and nurses, involved in patient care for that healthcare tenant.

The healthcare systems 322 can each include, for example, patient monitors and clinical systems for doctors, nurses, emergency personnel and/or other care providers, where such monitors or systems can provide, for example, logging, reporting, alerting and/or notification functions for any data related to the patients 302 (e.g., any of the inputs 127 and/or metrics 130 described relative to FIGS. 1A and 2). In addition, or alternatively, the healthcare systems 322 can each include, for example, systems for lab technologists, pharmacists and radiologists. In addition, or alternatively, the healthcare systems 322 can include other systems that support patient care and/or administration functions such as registration, scheduling, billing, combinations of the foregoing and/or the like.

For each of the healthcare tenants 310, the one or more data sources 321 can include data streams or datasets that can be received or processed by the healthcare systems 322 such as, for example, EMRs and/or prescriptions for the patients 302, analyte measurements from the analyte sensor systems 304 for the patients 302, and/or any of the inputs 127 or metrics 130 described relative to FIGS. 1A and 2. In various cases, the one or more data sources 321 can be updated, for example, by the HCPs 314, the healthcare systems 322, the analyte sensor systems 304, the IDR management system 340, and/or other components.

In general, the analyte sensor systems 304 can each operate as described relative to the analyte sensor system 104 of FIGS. 1A-B and 2. In particular, the analyte sensor systems 304 can each be configured to generate analyte measurements, as described previously, for a particular patient of the patients 302 of at least one of the healthcare tenants 310. In certain aspects, the analyte sensor systems 304 can each transmit the analyte measurements for such patient, via the network 308, to the IDR management system 340 for processing, to one or more of the healthcare systems 322 (e.g., corresponding to a healthcare facility where the patient is admitted) for reporting and/or alerting, to one or more of the user display devices 307 for reporting and/or alerting, and/or to other components or systems. In some cases, such transmissions can occur via one or more interfaces that are specific to the IDR management system 340 and/or the analyte sensor systems 304.

In the illustrated aspect, the IDR management system 340 can include an IDR engine 312 that can operate as described relative to the IDR engine 112 of FIG. 1A. In certain aspects, the IDR management system 340 and/or the IDR engine 312 can be implemented with hardware and/or software, including (optionally) virtual machines and/or containers. In an example, the IDR management system 340 can be implemented as a single management server. In another example, the IDR management system 340 can be implemented in a plurality of virtual or physical servers, which may or may not be geographically co-located. In some aspects, the IDR management system 340 and/or other aspects of the computing environment 300 can be hosted on a cloud system.

In certain aspects, features of the components of the IDR management system 340 can be made accessible over an interface, e.g., via the network 308, to the user display devices 307. The user display devices 307 can include any type of computing device, including systems such as desktops, laptops, tablets, smartphones, and smartwatches, to name a few. The user display devices 307 can be operated by users, such as the patients 302 and/or the HCPs 314. In some cases, the user display devices 307 can include, for example, the display device 107 described relative to FIG. 1A and/or the display devices 107a, 107b, 107c, and 107d described relative to FIG. 1B, where such devices can be associated with the patients 302 and/or the HCPs 314 of any of the healthcare tenants 310. In various aspects, for each of the healthcare tenants 310, alerts, reports and/or notifications can be presented to the patients 302 and/or the HCPs 314, as applicable, via the user display devices 307.

In general, the data store(s) 350 can include any information collected, stored or used by the IDR management system 340. For example, in various aspects, the data store(s) 350 can include, machine learning models for determining interference effects, algorithmic compensation models, one or more substance databases similar to the interferent database 113 of FIG. 1A, configuration settings (e.g., configurations for determining interference effects, interference responses, etc.), data received or collected from the analyte sensor systems 304, combinations of the same and/or the like. In certain aspects, data stored in the data store(s) 350 can take the form of repositories, flat files, databases, etc.

In more detail, the IDR engine 312 can predictively and/or proactively detect and dynamically handle substance interference with the analyte sensor systems 304. In certain aspects, for each healthcare tenant of the healthcare tenants 310, the IDR engine 312 can detect administrations of substances, or combinations of substances, to the patients 302, for example, from stored data in the data sources 321, and/or via user entry (e.g., entry by any of the HCPs 314 or a corresponding patient of the patients 302). In certain aspects, the IDR engine 312 can identify the substances as interferents, or as non-interferents, by searching the data store(s) 350 in similar fashion to the interferent database 113 of FIG. 1A. Likewise, the IDR engine 312 can generate interference effects and determine and execute interference responses based on the interference effects in the fashion described relative to FIGS. 1A-B and 2.

In various aspects, for each healthcare tenant of the healthcare tenants 310, the IDR engine 312 can determine and execute the interference responses in consideration of its dynamic and fast-paced environment. For example, if a given detected administration is a planned administration of the substance, the IDR engine 312 can, via the healthcare systems 322 of a corresponding healthcare tenant of the healthcare tenants 310, notify one or more of the HCPs 314 assigned to a corresponding patient of the patients 302, and/or a pharmacy associated with the corresponding patient, regarding the interference. The notification can include, for example, information related to the interference effect, for example, so that the prescription and/or plan of care can be reviewed for potential modification.

In another example, in an effort to prevent treatment decisions based on inaccurate or unreliable information, the IDR engine 312 can initiate or configure reporting and/or alerting to the HCPs 314 of each of the healthcare tenants 310. The configuration can include, for example, blinding or blocking reporting of the analyte sensor measurements (e.g., blocking from presentation to the HCPs 314), suppressing alerts based on the analyte sensor measurements, or reporting or alerting differently based on different interference effects, analyte measurements or amount of data available. In another example, the IDR engine 312 can cause interference notifications to be generated and presented that inform the HCPs 314 that the analyte measurements generated by corresponding analyte sensor systems of the analyte sensor systems 304 may be less accurate for a period of time (e.g., the interference duration) due to the administrations of the substances. In some cases, the interference notifications can provide information related to the interference effects, such as interference biases or interference durations.

In some aspects, the interference responses determined and executed by the IDR engine 312 can involve compensating for the interference. For example, the IDR engine 312 can initiate algorithmic compensation for the interference effects. The algorithmic compensation can include automatically adjusting the analyte measurements and presenting the adjusted analyte measurements to the HCPs 314 of the healthcare tenants 310. In some cases, the adjusted analyte measurements can be presented in place of the unadjusted measurements generated by the analyte sensor systems 304, while in other cases the adjusted analyte measurements can be presented in conjunction with the unadjusted measurements. In some aspects, the presentation of the adjusted analyte measurements can be accompanied by, or prefaced with, alerts or disclaimers to the effect that analyte measurements are being algorithmically adjusted to compensate for the interference. In addition, or alternatively, the adjusted analyte measurements can be presented to the HCPs 314 in a visually different way than unadjusted analyte measurements for emphasis (e.g., different color, font, location, etc.). In addition, or alternatively, by way of further example, the IDR engine 312 can compensate for the interference effect by removing interference using an anti-fouling approach as described in U.S. Provisional Application No. 63/542,631 and International Application No. PCT/US2024/048484.

FIG. 4 illustrates an example of a process 400 for detecting and dynamically handling substance interference. In some aspects, the process 400 can be executed, for example, by the IDR engine 312 of FIG. 3 and/or the IDR engine 112 of FIGS. 1A-B and 2. In addition, or alternatively, the process 400 can be executed, for example, by the analyte sensor system 104 of FIGS. 1A-B and/or by the analyte sensor systems 304 of FIG. 3. In addition, or alternatively, the process 400 can be executed, for example, by the application 106 of FIGS. 1A-B and 2. In addition, or alternatively, the process 400 can be executed generally by the user display device 307 of FIG. 3, and/or any of the display devices 107 of FIGS. 1A-B and 2. Although any number of systems, in whole or in part, can implement the process 400, to simplify discussion, the process 400 will be described generically in relation to an IDR engine, such as the IDR engine 112 of FIG. 1 or the IDR engine 312 of FIG. 3.

At block 402, the IDR engine detects an administration of a substance to a patient, such as the patient 102 of FIG. 1 or one of the patients 302 of FIG. 3. As described previously, in various aspects, the administration can be a planned administration (e.g., indicated in a stored prescription and/or by user entry) or a past or completed administration (e.g., indicated in an EMR and/or by user entry). In general, the administration of the substance can be detected in any of the ways described above relative to FIGS. 1A-B, 2, and 3. In some cases, a new prescription and/or new information in an EMR can trigger the detection at the block 402.

At block 404, the IDR engine determines substance administration information for the substance. The substance administration information can include, for example, a substance name (e.g., a medication name), dose, and/or time of administration. For example, in a non-ambulatory setting such as described relative to FIG. 3, some or all of the substance administration information can be retrieved from an EMR associated with the patient. In some aspects, the EMR details each substance that has been administered, and/or is planned for administration, to the patient. In these aspects, at least some of the substance administration information can be extracted from the EMR. In another example, some or all of the substance administration information can be extracted or retrieved from a stored prescription for the patient. In addition, or alternatively, some or all of the substance administration information can be entered by a patient and/or by an HCP such as any of the HCPs 314 of FIG. 3.

At block 406, the IDR engine determines an interference classification of the substance. As described relative to FIGS. 1A-B, 2 and 3, the IDR engine can identify the substance as an interferent, or as a non-interferent, based on the substance administration information and stored information regarding known interferents. The stored information regarding known interferents can include, for example, an interferent database such as the interferent database 113 of FIG. 1A. In certain aspects, the block 406 can include searching all or part of the substance administration information against the interferent database. If the search yields a match, the substance can be identified as an interferent. Otherwise, if the search does not yield a match, the substance can be identified as a non-interferent.

At decision block 408, the IDR engine determines whether the substance has been classified, or identified, as an interferent. If it is determined at the decision block 408 that the substance has not been classified, or identified, as an interferent, no further action is taken based thereon and the process 400 ends. Otherwise, if it is determined at the decision block 408 that the substance is an interferent, the process 400 proceeds to block 410.

At block 410, the IDR engine determines interference characteristics associated with the administration of the substance. For example, the IDR engine can extract at least some of the interference characteristics from an interferent database such as the interferent database 113 of FIG. 1A. For example, the interference characteristics can include pharmacokinetic, biochemical, and/or electrophysiological characteristics of the substance. In addition, or alternatively, the interference characteristics can include molecular weight (e.g., 151 grams per mole), standard dosage, aqueous solubility (e.g., high, low, etc.), a standard administration schedule (e.g., oral, every six hours), an indication of whether the substance is an electrochemical interferent, an indication of whether the substance is an enzyme interferent, and/or the like. In addition, or alternatively, the interference characteristics can include peak concentration in ISF (Cmax), time to reach Cmax (Tmax), elimination half-life, rate of absorption, rate of clearance, and/or the like. In various aspects, Cmax, Tmax, rate of absorption, rate of clearance and/or other characteristics can be determined or estimated by the IDR engine based on the substance administration information, the patient characteristics and/or other interference characteristics of the substance.

In some cases, the substance may be administered to the patient in combination with one or more other substances, for example, due to the patient's disease state or comorbidities. Accordingly, in various aspects, the interference characteristics determined at the block 410 can include multi-substance interference characteristics that consider, for example, an impact of the substance's combination with the other substance(s). More generally, the multi-substance interference characteristics can include additional and/or different interference characteristics relative to the interference characteristics that would be determined for an individual administration. In an example, the substance, due to its combination with the other substance(s), can be associated with a different indication of electrochemical and/or enzyme interference. In another example, the substance, due to its combination with the other substance(s), can be associated with a different Cmax, Tmax, elimination half-life, rate of absorption, rate of clearance, and/or the like. Other examples will be apparent to one skilled in the art after a detailed review of the present disclosure.

At block 412, the IDR engine generates an interference effect of the substance on an analyte sensor system of the patient, such as on the analyte sensor system 104 of FIGS. 1A-B and 2 and/or on any of the analyte sensor systems 304 of FIG. 3. The interference effect can be based on the substance administration information, the interference characteristics, and/or patient characteristics as described relative to FIGS. 1A-B and 2. For example, in certain aspects, the interference effect can be based on the multi-substance interference characteristics discussed relative to the block 410.

In various aspects, the interference effect can be, or can include, an interference bias and/or an interference duration. The interference bias can quantify, for example, a discrepancy between measured and actual values (e.g., analyte measurements are approximately 10% lower than actual analyte concentration levels as measured, for example, utilizing one or more intravenous and/or capillary-based analyte measurement systems such as a blood glucose meter, a blood gas analyzer, a biochemistry glucose and/or lactate analyzer, etc.). In some cases, the interference bias can be an output of a model or function that accepts, for example, an analyte measurement (e.g., a measured glucose value) as an input. The interference duration can indicate, for example, an amount of time following administration during which an interference threshold is satisfied. In various aspects, the interference threshold can be expressed, for example, in terms of a concentration of the substance, interference bias, and/or another suitable metric.

In some aspects, the interference effect can be generated in a rules-based fashion. In an example, the interference bias and the interference duration can be generated based on stored information (e.g., bench results) related to expected interference at particular concentrations of the substance. The stored information can be included, for example, in one or more databases such as the interferent database 113 of FIG. 1A and/or the data store(s) 350 of FIG. 3. In some aspects, the IDR engine can obtain, from the databases, data related to expected interference (e.g., interference biases, scaled interference levels, etc.) at particular concentrations of the substance. According to this example, the IDR engine can extrapolate the interference bias and the interference duration, from the obtained data, based on the Cmax, Tmax, elimination half-life, rate of absorption, rate of clearance, and/or other interference characteristics, including multi-substance interference characteristics as discussed previously.

In another example of a rules-based method, the interference bias can be generated based on a calculated current for a given concentration of the substance. For example, the IDR engine can perform theoretical calculations for an expected current to be generated based on the electrophysiological characteristics of the substance as well as Cmax, Tmax, elimination half-life, rate of absorption, rate of clearance and/or other interference characteristics, including multi-substance interference characteristics as discussed previously. In some cases, the expected current can be obtained from stored data, for example, in the interferent database 113 of FIG. 1A and/or the data store(s) 350 of FIG. 3. According to this example, the IDR engine can extrapolate the interference bias and the interference duration from the expected current and/or the Cmax, Tmax, elimination half-life, rate of absorption, rate of clearance, and/or other interference characteristics.

In addition, or alternatively, the IDR engine can generate the interference effect using a model that is at least partially based in machine learning (e.g., supervised learning). For example, the model can be trained on datasets for a large set of patients from one or multiple hospitals (e.g., for multiple of the healthcare tenants 310 described relative to FIG. 3), where the datasets can include information similar to the inputs 127 and the metrics 130 described relative to FIGS. 1A and 2. According to this example, the datasets on which the model is trained can include records detailing sets of features such as patient characteristics, substance characteristics, analyte sensor systems, hospitals or medical facilities, and/or the like. Each record can further include, or be labeled with, an interference effect (e.g., bias and/or duration) given the features of the record. Therefore, according to this example, for a given patient, the IDR engine can use the patient characteristics, the substance administration information, the interference characteristics (e.g., multi-substance interference characteristics) and/or other available information (e.g., any of the inputs 127 or metrics 130 of FIG. 2) to determine the interference effect using the example model described above.

At block 414, the IDR engine determines an interference response based on the interference effect determined at the block 412. In general, the interference response can include one or more defined actions that are determined in any of the ways described above relative to FIGS. 1A-B, 2, and 3. For example, the determined interference response can involve compensating for the effect in the analyte measurements (e.g., automatically adjusting the measurements or endeavoring to remove interference via an anti-fouling approach), configuring reporting or alerting (e.g., blinding or blocking reporting or suppressing alerting), reporting or alerting differently based on different interference effects, glucose levels or amount of data available, performing blocking of one or more glucose values, and/or taking other proactive actionable steps.

For example, the interference response can be configured to vary based on attaining specified thresholds related to the interference effect (e.g., interference bias and/or interference duration) and/or analyte sensor measurement ranges (e.g., glucose value ranges). Depending on the thresholds attained, alerts can be issued or algorithmic compensation can be activated. For example, if interference bias is expected to be above a customizable acceptable threshold set, for example, by a user or HCP, a customizable alert can be issued and algorithmic compensation can be activated. In another example, if interference bias is expected to be above a customizable acceptable threshold set, for example, by a user or HCP, for at least X number of readings (e.g., a configurable number set by a user or HCP), a customizable alert can be issued.

In some aspects, the interference response can be determined based on an amount of data available. Consider an example in which there is no quantification, or an insufficient quantification, of the interference effect to initiate algorithmic compensation (e.g., due to a lack of a data parameter needed to generate the interference bias and/or duration). In some aspects, the determined interference response can include generation of an alert to treat analyte sensor measurements (e.g., glucose values) with caution for the next X hours (e.g., a configurable number set by a user or HCP). In addition, or alternatively, the determined interference response can include blinding or blocking the analyte sensor measurements (e.g., glucose values) from being presented for the next X hours (e.g., a configurable number set by a user or HCP). In addition, or alternatively, the determined interference response can include generation of an alert that an interferent has been identified and that analyte sensor measurements (e.g., glucose values) are being algorithmically corrected to avoid inaccuracies.

Table 1 below illustrates example configurations for defined actions that can be included in the interference response.

TABLE 1

| | Interference | Data Sufficient | |
|---|---|---|---|
| Glucose | Bias | for Algorithmic | |
| Range | Threshold | Compensation? | Example Actions |
| <70 | 0-5% | Not Applicable | No action |
| mg/dL | | | |
| <70 | 5-10% | Not Applicable | Cautionary alert without |
| mg/dL | | | further action |
| <70 | >10% | NO | For example, one of the |
| mg/dL | | | two options below: |
| | | | Alert to interpret estimated |
| | | | glucose values (EGVs) with |
| | | | caution and to include |
| | | | expected direction of bias |
| | | | Blind data |
| <70 | >10% | YES | For example, one of the |
| mg/dL | | | three options below: |
| | | | Alert stating interferent |
| | | | present but EGVs have been |
| | | | adjusted |
| | | | Alert stating interferent |
| | | | present but EGVs only |
| | | | partially adjusted (e.g., |
| | | | observed interference is not |
| | | | fully compensated). The alert |
| | | | can advise that EGVs be |
| | | | interpreted with caution |
| | | | Blind data |

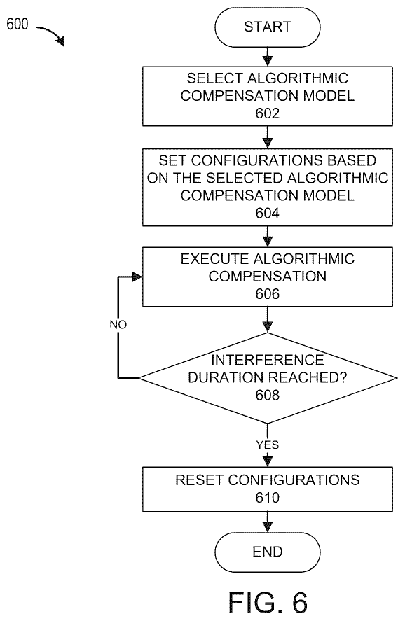

At block 416, the IDR engine executes, or causes execution, of the interference response. In general, the interference response can be executed, or caused to execute, in any of the ways described above relative to FIGS. 1A-B, 2, and 3. For example, the IDR engine can initiate configuration, compensation, alerting and/or reporting in correspondence to the interference response. An example of algorithmic compensation that can be initiated will be described relative to FIG. 6. After block 416, the process 400 ends.

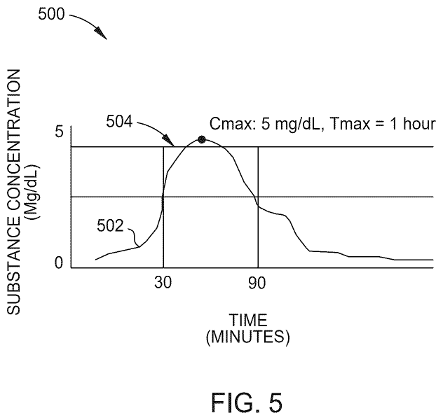

FIG. 5 is a graph 500 illustrating data that can be generated, for example, as part of generating an interference effect at the block 412 of FIG. 4. The graph 500 plots a substance concentration 502 in ISF over a time T beginning at a time of administration. In the illustrated aspect, an interference threshold is set at approximately 3 mg/dL. According to the example of FIG. 5, the substance concentration 502 remains at or above the interference threshold for an interference duration 504. In the example of FIG. 5, the interference duration 504 is approximately 60 minutes (i.e., approximately 30 to 90 minutes after administration).

FIG. 6 illustrates an example of a process 600 for implementing algorithmic compensation of analyte sensor measurements. In some aspects, the process 600 can be executed, for example, by the IDR engine 312 of FIG. 3 and/or the IDR engine 112 of FIGS. 1A-B and 2. In addition, or alternatively, the process 600 can be executed, for example, by the analyte sensor system 104 of FIGS. 1A-B and/or by the analyte sensor systems 304 of FIG. 3. In addition, or alternatively, the process 600 can be executed, for example, by the application 106 of FIGS. 1A-B and 2. In addition, or alternatively, the process 600 can be executed generally by the user display device 307 of FIG. 3, and/or any of the display devices 107 of FIGS. 1A-B and 2. Although any number of systems, in whole or in part, can implement the process 600, to simplify discussion, the process 600 will be described generically in relation to an IDR engine, such as the IDR engine 112 of FIG. 1 or the IDR engine 312 of FIG. 3.

At block 602, the IDR engine selects an algorithmic compensation model. The selected algorithmic compensation model can be utilized to compensate analyte measurements produce by an analyte sensor system such as, for example, the analyte sensor system 104 of FIGS. 1A-B and/or any of the analyte sensor systems 304 of FIG. 3. The analyte sensor system can be used (e.g., worn) by a patient as described previously.

In various aspects, different algorithmic compensation models can be selected at the block 602 for different scenarios. Examples of different algorithmic compensation models will be described below relative to compensation of glucose values. It should be appreciated, however, that other types of compensation models are likewise contemplated without deviating from the present disclosure. In some aspects, a single default algorithmic compensation model can be utilized for all compensation (e.g., a machine learning model), such that the selection at the block 602 can be omitted.

In some aspects, an algorithmic compensation model can be tailored to substances that are periodically administered throughout a day. Consider an example of a patient taking an acetaminophen tablet of 1,000 mg every 6 hours. If the IDR engine identifies the substance as an interferent at the given dose, interference characteristics associated with the administration may demonstrate peak concentrations are observed within 30 minutes to an hour and that substance concentration can remain for 5-6 hours in plasma. Accordingly, a corresponding algorithmic compensation model can call for initiating compensation approximately 15 minutes after a time of administration and continuing the compensation until approximately 3 hours after the time of administration. The corresponding algorithmic compensation model can call for the foregoing functionality to be repeated as the patient takes additional doses of acetaminophen throughout the day. If an EMR, for example, indicates that the patient has hepatic impediment, an elimination time of the substance can be lowered and the corresponding algorithmic compensation can instead be executed for 4 hours. The information used to perform the algorithmic compensation can be included, for example, in a database such as the interferent database 113 of FIG. 1A and/or the data store(s) 350 and/or in other stored data such as the EMR.

In some aspects, another algorithmic compensation model can be tailored to scenarios where interference bias is non-uniform across a range of analyte measurements, such as a glucose range. Consider an example in which a substance D1 is administered to the patient at time t1. According to this example, a database such as, for example, the interferent database 113 of FIG. 1A and/or the data store(s) 350 of FIG. 3, can show a match with the substance and flag the substance as an interferent. Further, the IDR engine can determine that the substance causes a positive interference bias (e.g., >10%) for glucose levels above Y mg/dL for an interference duration of T hours. According to this example, a corresponding algorithmic compensation model can track the glucose values and when the glucose values exceed Y, the corresponding algorithmic compensation model can trigger compensation for the interference period T.

According to the above example of non-uniform bias, the corresponding algorithmic compensation model can rely on analyte sensor parameters such as, for example, a sensitivity m of the analyte sensor system. In various aspects, the corresponding algorithmic compensation model can artificially mimic change in the sensitivity m by applying a factor q (e.g., on the order of the interference bias, such as 10%), where the new sensor sensitivity qm compensates for the substance's interference effect and leads to accurate glucose values above Y mg/dL for the interference duration T. The new sensor sensitivity qm is an example of an updated sensor parameter. As the interference period T approaches the end, the corresponding algorithmic compensation model can modify the factor q back to its previous value (e.g., 1), in order for the sensor sensitivity to return to m once the interference period ends.

In some aspects, various algorithmic compensation models can be tailored to scenarios where interference bias is uniform across a range of analyte measurements, such as a glucose range. Consider an example in which a substance D2 is flagged by a database such as, for example, the interferent database 113 of FIG. 1A and/or the data store(s) 350 of FIG. 3, as an interferent with electroactive material that causes a uniform bias across the entire glucose range. In other words, according to this example, the analyte sensor system senses such substances that lead to higher electrical current readings, and hence greater interference bias, without the presence of additional glucose. In various aspects, a corresponding algorithmic compensation model can compensate for such erroneous bias through application of scalar adjustment values that cancel the expected increase in glucose readings. For example, if the interference bias of the electroactive material is characterized to be on the order of +s for an interference duration associated with the administration of D2, a scalar adjustment value of −s mg/dL can be established. Thereafter, the scalar adjustment value of −s mg/dL can be applied to the measured glucose values.

Continuing the above example, another corresponding algorithmic compensation model can compensate for the erroneous bias via an analyte sensor parameter corresponding to a constant, additional current generated by the substance D2, for example. Equation 1 below illustrates an example formula that can be used to compensate a value X corresponding to EGV based on an analyte sensor parameter z. In the example of Equation 1, a total current Y equals a sum of X, b, and z, where b is a known background signal and z corresponds to the constant additional current generated by the substance D2, for example. In various embodiments, z can be calculated based on data retrieved from the interferent database 113 or, in some cases, can be retrieved from the interferent database 113. In this way, EGV, as represented by X, can be compensated (e.g., adjusted) based on the additional current generated by the substance D2 as represented by z.

$$Y = X + b + z \qquad Equation 1$$

In some aspects, another algorithmic compensation model can be tailored to patient physiology. Consider an example in which a substance drug D3 is administered to a patient and the substance is flagged as an interferent for its effect on changing the patient's glucose dynamics for an interference duration. This is a case where the drug's effect on physiology (and not direct interaction with the analyte sensor system) should be compensated. In this case, a corresponding algorithmic compensation model can update a state space of parameters of a glucose transport model between blood interstitial compartments. For example, if the substance D3 has been characterized to affect perfusion and increase the transfer of glucose from blood to the interstitial fluid, the corresponding parameters in the transport model can be updated to represent this increase and to lead to accurate glucose values during the interference period of drug D3.

With reference to the process 600, at block 604, the IDR engine sets configurations based on the selected algorithmic compensation model. For example, the IDR engine can temporarily adjust one or more sensor parameters (e.g., a sensor sensitivity parameter as discussed above), one or more parameters of a physiology model (e.g., parameters of a glucose transport model as discussed above), and/or the like. In another example, the IDR engine can establish a scalar adjustment value to use in adjustments (e.g., in the case of uniform bias as described above). In some aspects, if the selected algorithmic compensation model does not call for establishment or modification of any parameters, the block 604 can be omitted.

At block 606, the IDR engine executes, or causes execution of, the algorithmic compensation model. At decision block 608, the IDR engine determines whether an interference duration has been reached. If not, the process 600 returns to the block 606 to continue execution of the selected algorithmic compensation model. Otherwise, if it is determined at the decision block 608 that the interference duration has been reached, the process 600 proceeds to block 610. At block 610, the IDR engine resets the configurations to their previous status (e.g., their status prior to the block 604). In various aspects, if no configurations were set at the block 602, the block 610 can be omitted. After block 610, the process 600 ends.

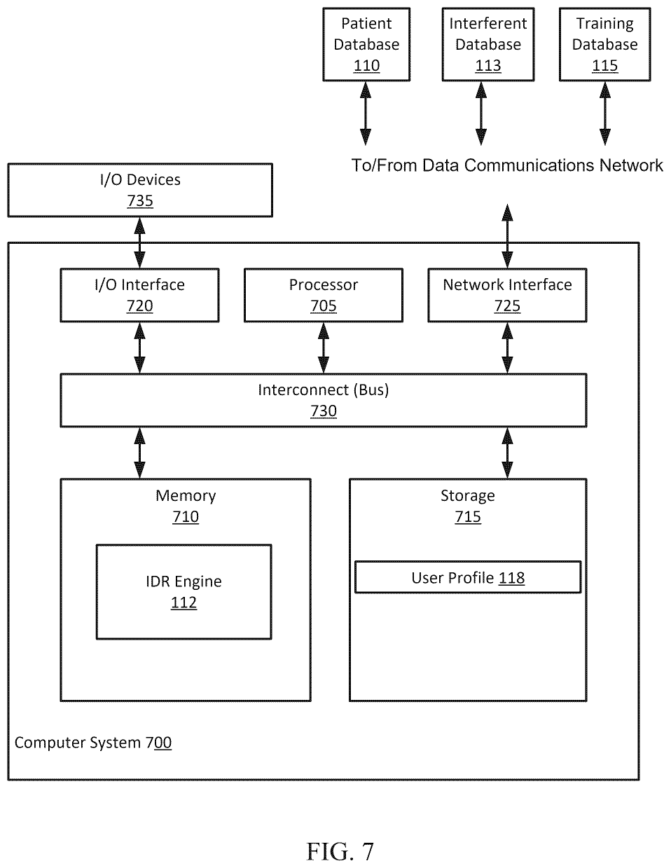

FIG. 7 is a block diagram depicting a computer system 700 configured for detecting and dynamically handling substance interference, according to certain aspects disclosed herein. In some aspects, the computer system 700 can correspond to, or be resident on, the analyte sensor system 104 of FIG. 1A, the display device 107 of FIG. 1A, the display devices 107a, 107b, 107c, and 107d described relative to FIG. 1B, the IDR management system 340 of FIG. 3, the analyte sensor systems 304 of FIG. 3, the user display devices 307 of FIG. 3, and/or the like.

Although depicted as a single physical device, in aspects, the computer system 700 can be implemented using virtual device(s), and/or across a number of devices, such as in a cloud environment. As illustrated, the computer system 700 includes a processor 705, a memory 710, a storage 715, a network interface 725, and one or more I/O interfaces 720. In the illustrated aspect, the processor 705 retrieves and executes programming instructions stored in the memory 710, as well as stores and retrieves application data residing in the storage 715. The processor 705 is generally representative of a single CPU and/or GPU, multiple CPUs and/or GPUs, a single CPU and/or GPU having multiple processing cores, and the like.

The memory 710 is generally included to be representative of a random access memory (RAM). The storage 715 can be any combination of disk drives, flash-based storage devices, and the like, and can include fixed and/or removable storage devices, such as fixed disk drives, removable memory cards, caches, optical storage, network attached storage (NAS), or storage area networks (SAN).

In some aspects, the I/O devices 735 (such as keyboards, monitors, etc.) can be connected via the I/O interface(s) 720. Further, via the network interface 725, the computer system 700 can be communicatively coupled with one or more other devices and components, such as the training database 115, the interferent database 113 and/or the patient database 110. In certain aspects, the computer system 700 is communicatively coupled with other devices via a network, which can include the Internet, local network(s), and the like. The network can include wired connections, wireless connections, or a combination of wired and wireless connections. As illustrated, the processor 705, memory 710, storage 715, network interface(s) 725, and the I/O interface(s) 720 are communicatively coupled by one or more interconnects 730. In certain aspects, the computer system 700 is representative of the display device 107 associated with the user. In certain aspects, as discussed above, the display device 107 can include the user's laptop, computer, smartphone, and the like. In another aspect, the computer system 700 is a server executing in a cloud environment.

In the illustrated aspect, the storage 715 includes the patient profile 118. The memory 710 includes the IDR engine 112. The IDR engine 112 can be executed by the computing system 700 to perform operations, for example, of the process 400 of FIG. 4 and/or the process 600 of FIG. 6.

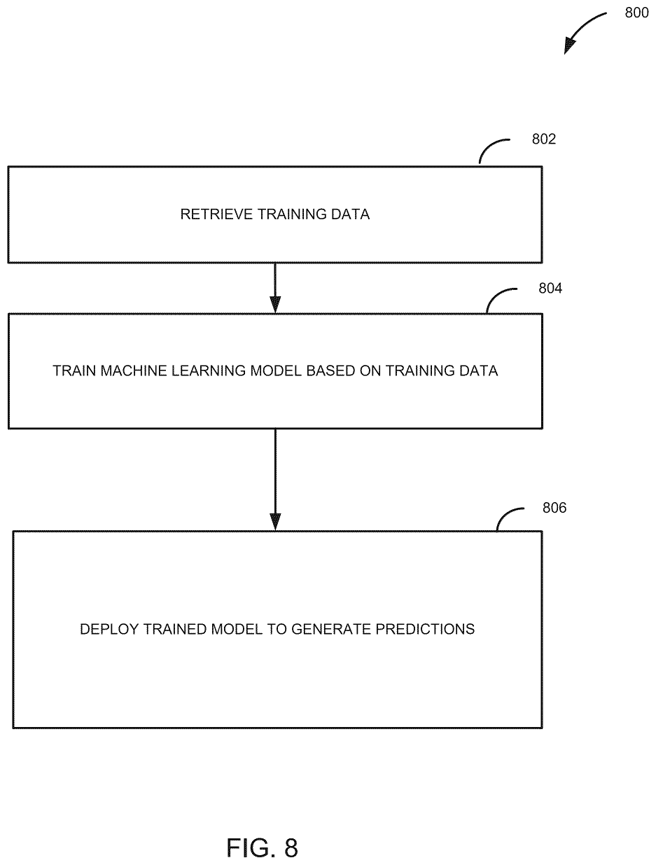

FIG. 8 is a flow diagram depicting a process 800 for training machine learning models to predict or determine whether a substance is interferent, to predict or determine an interference effect, and/or to predict or determine an interference response. Process 800 begins, at block 802, by training server system, such as training server system 125 illustrated in FIG. 1, retrieving data from a training database, such as training database 115 illustrated in FIG. 1. As mentioned previously, training database 115 can provide a repository data (e.g., from patient profiles) associated one or more patients (e.g., users or non-users of analyte sensor system 104 and/or application 106) and/or data associated with one or more substances (e.g., from interferent database 113).

Retrieval of data from training database 115 by training server system 125, at block 802, can include the retrieval of all, or any subset of, information maintained by training database 115. For example, where training database 115 stores information for 100,000 substance administrations (or for 100,000 patients), data retrieved by training server system 125 to train one or more machine learning models can include information for all 100,000 substance administrations (or all 100,000 patients) or only a subset of the data for those patients or administrations, e.g., data associated with only 50,000 administrations or only data from the last ten years.

As an illustrative example, integrating with on premises or cloud based medical record databases through Fast Healthcare Interoperability Resources (FHIR), web application programming interfaces (APIs), Health Level 7 (HL7), and or other computer interface language can enable aggregation of healthcare historical records for baseline assessment in addition to the aggregation of de-identifiable patient data from a cloud based repository. Similarly, when integrating into the medical record databases, the integration can be accomplished by directly interfacing with the electronic medical record system or through one or more intermediary systems (e.g., an interface engine, etc.).

As an illustrative example, at block 802, training server system 125 can retrieve information for 100,000 substance administrations stored in training database 115 to train a model to predict, for a given patient and substance, an interferent classification of the substance, interferent effect of the substance, an interference response, a manner of executing an interferent response, etc. Each of the administrations can have a corresponding data record that is further associated with a patient (e.g., based on a corresponding patient profile 118 as discussed relative to FIGS. 1A and 2) stored in training database 115.

The training server system 125 then uses information in each of the records to train an artificial intelligence or ML model (for simplicity referred to as “ML model” herein). Examples of types of information included in the records were provided above relative to FIG. 1A. The information in each of these records can be featurized (e.g., manually or by training server system 125), resulting in features that can be used as input features for training the ML model as discussed relative to FIG. 1A. Features used to train the machine learning model(s) can vary in different aspects.

At block 804, process 800 continues by training server system 125 training one or more machine learning models based on the features and labels associated with the training data. In some aspects, the training server does so by providing the features as input into a model. This model can be a new model initialized with random weights and parameters, or can be partially or fully pre-trained (e.g., based on prior training rounds). Based on the input features, the model-in-training generates some output, such as an interferent classification of a substance, an interferent effect of the substance, an interference response, a manner of executing an interferent response, etc.

In certain aspects, training server system 125 compares this generated output with the actual label associated with the corresponding historical patient record to compute a loss based on the difference between the actual result and the generated result. This loss is then used to refine one or more internal weights and parameters of the model (e.g., via backpropagation) such that the model learns to predict, for example, for a given patient and substance, an interferent classification of the substance, interferent effect of the substance, an interference response, a manner of executing an interferent response, etc.

One of a variety of machine learning algorithms can be used for training the model(s) described above. For example, one of a supervised learning algorithm, a neural network algorithm, a deep neural network algorithm, a deep learning algorithm, etc. can be used.

At block 806, training server system 125 deploys the trained model(s) to make predictions during runtime. In some aspects, this includes transmitting some indication of the trained model(s) (e.g., a weights vector) that can be used to instantiate the model(s) on another device. For example, training server system 125 can transmit the weights of the trained model(s) to IDR engine 112, which could execute on display device 107, etc. The model(s) can then be used to determine, in real-time, for a given patient and substance, an interferent classification of the substance, interferent effect of the substance, an interference response, a manner of executing an interferent response, etc. In certain aspects, the training server system 125 can continue to train the model(s) in an “online” manner by using input features and labels associated with new patient records.

Further, similar methods for training illustrated in FIG. 8 can also be used to train models using patient-specific records to create more personalized models for making predictions associated interference detection and response. For example, a model trained based on population data can be re-trained after deployment. For example, the model can be re-trained after the model is deployed for a specific patient to create a more personalized model for the patient. Since the personalized model is based, at least in part, on the patient's own data the patient's own inputs 128 and metrics 130 as discussed relative to FIGS. 1A and 2), it may be able to more accurately make predictions on an interferent classification of a substance, interferent effect of the substance, an interference response, a manner of executing an interferent response (e.g., by adjusting measurements more accurately), etc.

According to an embodiment, a method of dynamically handling substance interference is performed by a computer system. The method includes detecting an administration of a substance to a user of an analyte sensor system. The method also includes identifying the substance as an interferent with the analyte sensor system based on information related to the administration of the substance. The method also includes, responsive to the identifying, generating an interference effect of the substance on the analyte sensor system based on the information related to the administration of the substance. The method also includes determining an interference response based on the interference effect. The method also includes executing the interference response in relation to the analyte sensor system.

The information related to the administration of the substance may include a dose and a time of administration.

The method may include extracting at least some of the information related to the administration of the substance from an electronic medical record associated with the user.

The identifying the substance as an interferent may include searching at least a portion of the information related to the administration of the substance against a database that includes a list of substances that pose a risk of interference to the analyte sensor system.

The method may include determining interference characteristics associated with the administration of the substance, where the interference effect is determined based on the interference characteristics. The interference characteristics may include at least one of a pharmacokinetic, biochemical, or electrophysiological characteristic of the substance. The interference characteristics may include at least one of peak concentration, time to reach peak concentration, elimination half-life, rate of absorption, or rate of clearance. The substance may be administered to the user in combination with at least one other substance, and the determining interference characteristics may include determining at least one interference characteristic based on an impact of the combination of the substance with the at least one other substance.

The interference effect may include: an interference bias, the interference bias quantifying a discrepancy between analyte measurements generated by the analyte sensor system and actual analyte concentration levels; and an interference duration, the interference duration indicating an amount of time for which an interference threshold is satisfied. The generating an interference effect may include extrapolating the interference bias and the interference duration, from stored information related to expected interference at particular concentrations of the substance, based on an estimated peak concentration of the substance and at least one of a rate of absorption or a rate of clearance of the substance. The generating an interference effect may include extrapolating the interference bias and the interference duration, from information related to an expected current to be generated at particular concentrations of the substance, based on an estimated peak concentration of the substance and at least one of a rate of absorption or a rate of clearance of the substance.

The detecting may include detecting a planned administration of the substance to the user of the analyte sensor system based on stored information related to the user.

The detecting may include detecting a completed administration of the substance to the user of the analyte sensor system based on stored information related to the user.

The interference response may include algorithmic compensation of analyte sensor measurements generated by the analyte sensor system, and the executing may include initiating the algorithmic compensation.

The executing the interference response may include alerting at least one of a healthcare professional or the user of the interference effect.

The executing the interference response may include blocking analyte measurements from the analyte sensor system from presentation to at least one of healthcare personnel or the user for a defined duration.

The executing the interference response may include initiating algorithmic compensation for the interference effect for a defined duration based on a model.

The executing the interference effect may include automatically adjusting analyte measurements generated by the analyte sensor system based on the model. The executing may include presenting the automatically adjusted analyte measurements to at least one of the user or a healthcare professional in place of the analyte measurements generated by the analyte sensor system. In some aspects, the model may be tailored to compensate for interference bias that is uniform across an analyte measurement range, the initiating algorithmic compensation may include establishing a scalar adjustment value based on the interference effect, and the automatically adjusting may include applying the scalar adjustment value to the analyte measurements generated by the analyte sensor system. In some aspects, the model may be tailored to compensate for interference bias that is non-uniform across an analyte measurement range and the initiating algorithmic compensation may include temporarily adjusting a sensor sensitivity parameter of the analyte sensor system. In some aspects, the model may be tailored to patient physiology, and the initiating algorithmic compensation may include temporarily adjusting a parameter of a glucose transport model.

The executing the interference response may include implementing an anti-fouling approach to compensate for the interference effect.

The executing the interference response may be based on attaining one or more thresholds in relation to the interference effect.

The substance may include a medication.

The analyte sensor system may include a continuous glucose monitor.

The analyte sensor system may include the computer system.

The computer system may be a mobile device in communication with the analyte sensor system.

According to another embodiment, a system for dynamically handling substance interference includes memory and a processor in communication with the memory. The memory includes executable instructions. The processor is configured to execute the instructions to: detect an administration of a substance to a user of an analyte sensor system; identify the substance as an interferent with the analyte sensor system based on information related to the administration of the substance; responsive to the identification, generate an interference effect of the substance on the analyte sensor system based on the information related to the administration of the substance; determine an interference response based on the interference effect; and execute the interference response in relation to the analyte sensor system.

According to another embodiment, a computer-program product includes a non-transitory computer-usable medium having computer-readable program code embodied therein. The computer-readable program code is adapted to be executed to implement a method. The method includes detecting an administration of a substance to a user of an analyte sensor system. The method also includes identifying the substance as an interferent with the analyte sensor system based on information related to the administration of the substance. The method also includes, responsive to the identifying, generating an interference effect of the substance on the analyte sensor system based on the information related to the administration of the substance. The method also includes determining an interference response based on the interference effect. The method also includes executing the interference response in relation to the analyte sensor system.

Each of these non-limiting examples can stand on its own or can be combined in various permutations or combinations with one or more of the other examples.

The above detailed description includes references to the accompanying drawings, which form a part of the detailed description. The drawings show, by way of illustration, specific aspects in which the invention can be practiced. These aspects are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round”, a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) can be used in combination with each other. Other aspects can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter may lie in less than all features of a particular disclosed aspect. Thus, the following claims are hereby incorporated into the Detailed Description as examples or aspects, with each claim standing on its own as a separate aspect, and it is contemplated that such aspects can be combined with each other in various combinations or permutations. The scope of the invention should be determined with reference to the appended claims, along with the full scope of equivalents to which such claims are entitled.

What is claimed is:

1. A method of dynamically handling substance interference, the method comprising, by a computer system:
detecting an administration of a substance to a user of an analyte sensor system;
identifying the substance as an interferent with the analyte sensor system based on information related to the administration of the substance;
responsive to the identifying, generating an interference effect of the substance on the analyte sensor system based on the information related to the administration of the substance;
determining an interference response based on the interference effect; and
executing the interference response in relation to the analyte sensor system.

2. The method of claim 1, wherein the information related to the administration of the substance comprises a dose and a time of administration.

3. The method of claim 1, further comprising extracting at least some of the information related to the administration of the substance from an electronic medical record associated with the user.

4. The method of claim 1, wherein the identifying the substance as an interferent comprises searching at least a portion of the information related to the administration of the substance against a database comprising a list of substances that pose a risk of interference to the analyte sensor system.

5. The method of claim 1, further comprising determining interference characteristics associated with the administration of the substance, wherein the interference effect is determined based on the interference characteristics.

6. The method of claim 5, wherein the interference characteristics comprise at least one of a pharmacokinetic, biochemical, or electrophysiological characteristic of the substance.

7. The method of claim 5, wherein the interference characteristics comprise at least one of peak concentration, time to reach peak concentration, elimination half-life, rate of absorption, or rate of clearance.

8. The method of claim 5, wherein the substance is administered to the user in combination with at least one other substance, and the determining interference characteristics comprises determining at least one interference characteristic based on an impact of the combination of the substance with the at least one other substance.

9. The method of claim 1, wherein the interference effect comprises:
an interference bias, the interference bias quantifying a discrepancy between analyte measurements generated by the analyte sensor system and actual analyte concentration levels; and
an interference duration, the interference duration indicating an amount of time for which an interference threshold is satisfied.

10. The method of claim 9, wherein the generating an interference effect comprises extrapolating the interference bias and the interference duration, from stored information related to expected interference at particular concentrations of the substance, based on an estimated peak concentration of the substance and at least one of a rate of absorption or a rate of clearance of the substance.

11. The method of claim 9, wherein the generating an interference effect comprises extrapolating the interference bias and the interference duration, from information related to an expected current to be generated at particular concentrations of the substance, based on an estimated peak concentration of the substance and at least one of a rate of absorption or a rate of clearance of the substance.

12. The method of claim 1, wherein the executing the interference response comprises initiating algorithmic compensation for the interference effect for a defined duration based on a model.

13. The method of claim 12, wherein the executing the interference effect comprises automatically adjusting analyte measurements generated by the analyte sensor system based on the model.

14. The method of claim 13, wherein the executing comprises presenting the automatically adjusted analyte measurements to at least one of the user or a healthcare professional in place of the analyte measurements generated by the analyte sensor system.

15. The method of claim 13, wherein:
the model is tailored to compensate for interference bias that is uniform across an analyte measurement range;
the initiating algorithmic compensation comprises establishing a scalar adjustment value based on the interference effect; and
the automatically adjusting comprises applying the scalar adjustment value to the analyte measurements generated by the analyte sensor system.

16. The method of claim 13, wherein:
the model is tailored to compensate for interference bias that is non-uniform across an analyte measurement range; and
the initiating algorithmic compensation comprises temporarily adjusting a sensor sensitivity parameter of the analyte sensor system.

17. The method of claim 13, wherein:
the model is tailored to patient physiology; and
the initiating algorithmic compensation comprises temporarily adjusting a parameter of a glucose transport model.

18. The method of claim 1, wherein the executing the interference response comprises implementing an anti-fouling approach to compensate for the interference effect.

19. A system for dynamically handling substance interference, the system comprising:
a memory comprising executable instructions;
a processor in communication with the memory and configured to execute the instructions to:
detect an administration of a substance to a user of an analyte sensor system;
identify the substance as an interferent with the analyte sensor system based on information related to the administration of the substance;
responsive to the identification, generate an interference effect of the substance on the analyte sensor system based on the information related to the administration of the substance;
determine an interference response based on the interference effect; and
execute the interference response in relation to the analyte sensor system.

20. A computer-program product comprising a non-transitory computer-usable medium having computer-readable program code embodied therein, the computer-readable program code adapted to be executed to implement a method comprising:
detecting an administration of a substance to a user of an analyte sensor system;
identifying the substance as an interferent with the analyte sensor system based on information related to the administration of the substance;
responsive to the identifying, generating an interference effect of the substance on the analyte sensor system based on the information related to the administration of the substance;
determining an interference response based on the interference effect; and
executing the interference response in relation to the analyte sensor system.
